(19) United States
(12) Patent Application Publication
(10) Pub. No.: US 2022/0308534 A1
(43) Pub. Date: Sep. 29, 2022

(54) SYSTEM AND METHOD FOR SIMULATING A CHEMICAL OR BIOCHEMICAL METHOD

(71) Applicant: YF1, Nancy (FR)

(72) Inventors: Roger-Marc NICOUD, Lay-Saint-Christophe (FR); Etienne BERGER, NANCY (FR)

(73) Assignee: YF1, Nancy (FR)

(21) Appl. No.: 17/635,567

(22) PCT Filed: Aug. 24, 2020
(86) PCT No.: PCT/EP2020/073606

§ 371 Date: Feb. 15, 2022

(30) Foreign Application Priority Data
Aug. 29, 2019 (EP) 19306046.4

(51) Int. Cl. G05B 13/04; G16C 20/10; G16C 20/70

(52) U.S. Cl. CPC G05B 13/044; G16C 20/10; G16C 20/70

(57) ABSTRACT

The present invention relates to a system for the computer simulation of a chemical process comprising a plurality of functional modules for completing respective simulation levels of said chemical process, a storage module for storing experimental data relating to chemical species in a data structure that can be used by at least one functional module, a performance evacuation module, in which said process is defined by a set of files shared by ail the modules of the system, each file comprising a description of a raw material and a description of a decomposition of this raw material into chemical species, said files being the inputs and the outputs of said modules of the system, the decomposition into chemical species being preserved throughout the processing operations.

### TECHNICAL FIELD

The present invention relates to the field of modelling and simulation in process engineering. In particular, the present invention relates to a system and a process for computer-implemented simulation of a chemical or biochemical process.

### PRIOR ART

Fine chemical or biotechnology manufacturers should frequently estimate or compare production processes involving several operations and various pieces of equipment based on a very limited amount of information. This is particularly the case in the early stages of development. It should be noted that in these industries, when a production process should be devised, it often happens that the chances of commercialising the targeted molecule are very low, for example, for reasons pertaining to clinical trial failures. Hence, there are quite many technical and economic estimates to be made on many molecules that will be produced only in small amounts and for a limited period of time.

When manufacturers simply wish to obtain a quick simulation of a chemical or biochemical process in the absence of any knowledge of the internal state of the equipment implementing the process, managers and engineers estimate the performances of the various operations using empirical knowledge or even just intuition. This knowledge can be: the content of a compound A at the output of the reactor is 50% without this content being related to any knowledge of the internal conditions of the reactor or else the rate of recovery of a compound A in the separator is 95% without this content being related to any knowledge of the internal conditions of the separator. This fixed information allows relating the output amounts (also called final transient-state amounts; hereafter reference will be made indifferently to the terms final amounts or output amounts) to the input amounts (also called initial transient-state amounts; hereafter reference will be made indifferently to the terms initial amounts or input amounts). This method does not involve material or heat balances and therefore does not consider the internal states of the equipment. Hence, it cannot predict any dynamics. Starting from the knowledge of the input amounts of the process, it is possible to compute step-by-step, for each operation, the output amounts. Hence, it is possible to determine, using a simple calculator such as an Excel spreadsheet, the output amounts obtained upon completion of the process.

This method has the advantage of computational simplicity and speed. However, it has several drawbacks:

the number of possible scenarios is limited to the experimental knowledge of the operators, optimisations and detailed comparisons between the processes are not therefore possible;

when many operations take place in series during the process, the simulation becomes particularly complex and inaccurate. This complexity is increased in case of a compound recycling loop during the process;

transient modes are not taken into account. This method is limited to continuous systems or discontinuous batch systems wherein transients are not taken into account. Yet, quite often, the amounts produced by the fine chemical or biotechnology industries do not allow for using continuous production (unlike the petrochemical industries).

When manufacturers wish to know more accurately the final amounts and performances of a process, it is necessary to use proven process engineering methods, based on detailed modelling, such as Honeywell UNISIM®, Aspen HYSYS®. These models require the knowledge of a very large number of physicochemical parameters, in particular thermodynamics, which allow simulating the behaviour and performances of the system according to the internal conditions. These models take into account the material and heat balances and determine the evolution of internal state variables in the equipment. Although more accurate, this method is merely used by fine chemical or biotechnology manufacturers because it requires the consideration of a large number of internal state variables and resorting to numerical methods that are very complex for a non-specialist. The time and resources required to determine the physicochemical parameters and to develop the simulation tool are too important for a process that might subsequently only be used for very small amounts.

To date, none of the existing approaches is satisfactory. They are either limited to particularly simplistic scenarios, or too complex and require the determination of a very large number of parameters.

Hence, there is a need for a system and a method for the computer simulation of a chemical or biochemical process allowing the simulation of complex scenarios, including several operations, within a reasonable time while limiting the number of physico-chemical parameters required to per form the simulation. In particular, there is a need for an industrial tool, currently non-existent, allowing for cooperation between various stakeholders in the sizing and implementation of complex chemical or biochemical installations.

The present invention falls within this context.

### SUMMARY OF THE INVENTION

The embodiments of the invention offer an industrial tool for fine chemical process engineering. A functional technical feature of the embodiments lies in the simulation of processes for subsequent implementation hereof in production facilities for producing chemicals or biochemicals.

The embodiments allow forecasting, in a concrete way, the behaviour of chemical or biochemical processes to make some estimates, in particular on their industrial feasibility. The embodiments of the invention thus allow directing the development of chemical or biochemical processes with enough accuracy, and within reasonable time and cost, to allow estimating the chances of success of their industrial implementation, even before the set-up of an installation and the commissioning of this installation.

Therefore, the embodiments of the invention offer the industrial tool that is lacking today in the development of chemical or biochemical installations. They allow determining, well ahead, the industrial feasibility or the possible difficulties in implementing chemical or biochemical processes or installations.

A purely intellectual implementation of a simulation of chemical or biochemical processes is not possible. This is particularly the case of simulations for industrial needs. In practice, it is impossible to carry out the computations necessary to end up with sufficiently accurate and significant results allowing industrial decisions to be made, such as the installation of chemical installations. Furthermore, the purely intellectual implementation of the simulation would require a prohibitive time, in particular to calibrate the parameters of the model.

Yet, without a computer-assisted simulation, it is impossible to conduct predictive tests or to make a choice among a plurality of chemical or biochemical process projects of those that offer the best performance, and that, within an industrially reasonable time.

There is no purely intellectual, mathematical or even theoretical method that could comprehensively and quickly forecast the behaviour of a fine chemical process.

An object of the invention is to enable fine chemical and biotechnology industries to use relevant simulators by providing a tool that could be used by a non-expert in simulation, which allows a manufacturing project to be taken into account with physico-chemistry knowledge that is initially almost non-existent.

This tool should be able to assist the user in identifying the delicate or limiting points of the assessment process in order to determine the knowledge to acquire or to use in order to achieve a given objective, refining these forecasts as new knowledge becomes available. This tool should also be reliable, easy to use, and provide an accurate estimate of the technical and economic performances of the process studied which is adapted to the considered problem and could evolve according to the needs and the evolution of the industrial project.

The embodiments of the invention are based on a simulation taking into account the physico-chemical characteristics of the reagents and intermediates used in the simulated chemical or biochemical process or installation.

To this end, according to a first aspect, the invention relates to a system according to claim 1.

### Definitions

By “fine chemicals” also called “specialty chemicals”, it should be understood in this document a branch of the chemical industry that synthesises specific products, in low production volumes, but with high added value, and meeting high technical constraints, for example purity.

By “biotechnology”, it should be understood technologies for the production of molecules by fermentation, cell cultures, extraction from the natural environment.

By “algebraic equation”, it should be understood an equation having one or several unknown real variable(s).

By “differential equation”, it should be understood an equation having one or several unknown function(s); it is in the form of a relationship between these unknown functions and their successive derivatives.

By “explicit equation”, it should be understood an equation between different variables where one variable is expressed explicitly in terms of other variables.

By “implicit equation”, it should be understood an equation between different variables where no variable is expressed in terms of other variables.

By “internal state variable”, it should be understood a chemical variable that describes what is occuring inside a given piece of equipment, for example temperature, pressure, concentration . . . .

By “input state variable” or “output state variable”, it should be understood a chemical variable that describes what is being input into or output from, respectively, a given piece of equipment, for example temperature, pressure, concentration . . . .

By “pseudo internal state variable”, it should be understood an artificial variable enabling the transformation of simple “explicit algebraic equations” relating an output to an input in a mathematical formalism identical to that of predictive models using “differential-algebraic equations”.

By “predictive model”, it should be understood the association of a certain number of algebraic and differential equations based on concepts of chemistry and/or physics, the resolution of which enables the determination of the internal state variables of at least one operation performed in at least one piece of equipment.

Ry “operation”, it should be understood a transformation allowing switching from an input state into an output state (or from an initial state into a final state). In general, a chemical or biochemical process comprises a plurality of operations.

### DESCRIPTION OF THE FIGURES

FIG. 1 illustrates a chemical process implementing several operations O1, O2, O3, O4 and several pieces of equipment E1, E2, E3, E4.

FIG. 2 schematically illustrates the system in accordance with an embodiment of the invention.

FIG. 3 is a diagram illustrating steps for performing the simulation in accordance with an embodiment of the invention.

FIG. 4 represents a block diagram representing a device for implementing one or several embodiment(s) of the invention.

FIG. 5 represents a reaction diagram comprising a reactor with an input In and two outputs Out1, Out2.

FIG. 6 schematically illustrates a system 600 according to some embodiments.

FIG. 7 illustrates a design mode of the prior art.

FIG. 8 illustrates the treatments in a system according to the invention.

FIG. 9 is a flowchart of the traditional DVL purification process.

FIG. 10 is a flowchart of the DVL purification process with recycling of the extraction solvent.

FIG. 11 is a flowchart of the DVI purification process by thermal neutralisation of peroxide,

FIG. 12 is an equipment diagram of the DVL purification process with recycling of the extraction solvent,

FIG. 13 is an equipment diagram of the DVI purification process by thermal neutralisation of peroxide.

FIG. 14 is a flowchart of the sertraline-tetralone production process without racemisation.

FIG. 15 is a flowchart of the sertraline-tetralone production process with racemisation.

FIG. 16 is a flowchart of the sertraline-tetralone production process with racemisation and recycling of racemisation acetonitrile.

FIG. 17 is an equipment diagram of the sertraline-tetralone production process without racemisation.

FIG. 18 is an equipment diagram of the sertraline-tetralone production process with racemisation and recycling of the racemisation acetonitrile.

### DETAILED DESCRIPTION

A chemical process could be divided into a plurality of operations carried out in a plurality of pieces of equipment. The number of operations could be less than, greater than or equal to the number of pieces of equipment. Indeed, the number of operations could be greater than the number of pieces of equipment if several operations are performed in the same piece of equipment whereas the number of pieces of equipment could be larger than the number of operations if an operation requires several pieces of equipment.

FIG. 1 schematically illustrates these different cases with various operations O1 to O4 and various pieces of equipment E1 to E4 to implement these operations. For example, the operations may consist of mixtures, separations and various reactions. For example, the equipment may consist of reactors, mixers, extractors, filters, evaporators, etc. in this diagram, a raw material is received by the equipment E1 to perform an operation O1. The product derived from the equipment 1 (which is not only the result of the operation O1 as seen hereinafter), is supplied to the equipment E2 to perform the operation O2. A feedback loop exists between the pieces of equipment E2 and E1. Thus, the product derived from the equipment E2 is supplied to both the equipment E1 and the equipment E3. Hence, an additional operation O3 is also carried out in the equipment E1. Because of the feedback loop, the operations O1 and O3 are therefore carried out within the same equipment E1. The product derived from the equipment E2 is also supplied to the equipment E4, Thus, the operation O4 is carried out using two pieces of equipment E3 and E4. For example, E3 may be a reactor and the equipment E4 a heat exchanger.

The chemical process and the corresponding installation illustrated in FIG. 1, require two main steps to be industrially validated, for example in order to put the product derived from the process into production or to launch the construction of an installation. First, the designer and in fact, an entire design team grouping together chemists and also non-chemists (for example for the financial aspects which is obviously considered in an industrial design), should be provided with a tool allowing to define this process and this installation. Secondly, it should provide him (them) with a means of forecasting, i.e., simulating, various criteria enabling him (them) to assess the industrial feasibility of the project. Such a forecast or simulation also includes the possibility of comparing different processes or installations with each other in order to make an optimal choice.

The tool allowing all this should be effective in terms of accuracy but also in terms of ease of use and computation time. Thus, accuracy is not always the only relevant parameter in the design because in the very early stages of development, it should be ensured at first that it is relevant to go on with a complete feasibility study. Thus, in the first stages, rough estimates could be accepted, if these are enough for a decision to be made on the advisability of carrying on.

In a first step, the simulation mode used according to some embodiments is described hereinafter. In a second step, an overall system is described, allowing implementing this simulation mode. In a third step, examples of use of this system and an example of simulation are described.

The Simulation Modes

An operation (i) and a piece of equipment (j) allow switching from an input state (Yₑ^{i/j}) into an output state (Yₛ^{i/j}). The input state and output state variables are vectors representing chemical amounts at the input and at the output of an operation and of a piece of equipment of a chemical or biochemical process.

When a simple and quick simulation of an operation and of a piece of equipment is desired, the operation and the piece of equipment are described by explicit algebraic equations relating the input states (Yₑ^{i/j}) and the output states (Yₛ^{i/j}) for the operation (i) and of the piece of equipment (j):Yₛ^{i/j}=Gₑ(Yₑ^{i/j}). There is no predictive ambition in this approach.

In the case where a more complete and accurate simulation is desired, it is necessary to determine what is happening inside the equipment, and therefore to determine a vector of internal state variables (X^{i/j}) for an operation (i) and a piece of equipment (j). The internal state variables are vectors representing internal chemical quantities of an operation and of a piece of equipment of a chemical or biochemical process.

In fine chemistry and/or biotechnology, the equipment generally operates in a transient state, the internal state variables are therefore most often solutions of systems of algebraic and differential equations (differential-algebraic system) that represent material, heat balances, or thermodynamic equilibria, for example.

In the general case, the operation and the piece of equipment are described by a system of differential equations involving the internal state variables. These equations relate the vector of internal state variables (X^{i/j}) to the vector of input state variables (Yₑ^{i/j}) for the operation (i) and the piece of equipment (j):

$${P}_{RT}^{E} ( {Y}_{e}^{i / j} , t , {X}^{i / j} , \frac{{dX}^{i / j}}{dT} ) = 0.$$

The vector of output state variables (Yₛ^{i/j}) which could depend on time is then determined explicitly from the knowledge of the internal state variables:

Yₛ^{i/j}(t)=P_{RT}^{E}(t, X^{i/j}) which, in turn, depends on the vector of input state variables.

It should be noted that in the steady mode, the final state of the vector of output state variables is obtained immediately by Yₛ^{i/j}(t=∞)=P_{RT}^{E}(t=∞, X^{i/j})

where t_{∞} represents the end time of the operation in a discontinuous system or a sufficiently long time in a continuous system.

The equations describing the operations and the pieces of equipment of a chemical or biochemical process must be solved in a coupled way while taking into account the connections between all the operations and all the pieces of equipment throughout the entire process. In the case of a system of equations involving internal state variables, this solving might be particularly long due to convergence problems but above all requires the knowledge of physico-chemical parameters the determination of which might be long and difficult.

Two operation/pieces of equipment simulation modes are thus available. A quite trivial one relies on simple, explicit algebraic relationships between output and input state variables. The other one, much more accurate and predictive, requires solving of complex differential-algebraic equations and the knowledge of numerous physico-chemical parameters. Hence, it is desirable to be able to switch from a detailed approach using equations involving the internal state variables into a simplified approach using explicit algebraic equations without the intervention of internal state, variables for different operations within the same process. Thus, it would be possible to save precious time by choosing to simulate within a chemical or biochemical process an operation that is simple or has a secondary influence using an explicit algebraic equation and a more complex or critical operation using a differential-algebraic system involving the internal state variables. However, due to the mathematical structure of the equations, it is not possible to integrate these two types of equations in the same general solving system.

To enable the integration of these two types of equations in a single solving system, the inventors had the idea of replacing the explicit algebraic equations with differential-algebraic equations involving pseudo internal state variables. The differential-algebraic equations thus created are affected by a very small time constant in comparison with the characteristic time of the operation. It should be noted that a person skilled in the art knows the characteristic time of each operation of the process and will be able to choose a time constant that is low in comparison with this characteristic time (for example a time constant in the range of one second). By choosing a low time constant, for a time much greater than this time constant, the differential term vanishes in the steady-state mode and all what remains is to ensure that the other terms of the differential equation converge towards the conditions of the explicit algebraic equation.

Thanks to this transformation, the explicit algebraic equations could be integrated into the simulation system and the user could choose between a fine modelling of each operation or not. Thus, while switching from an algebraic system into a differential-algebraic system might seem to make the simulation more complex, it actually allows flexibility in the simulation without jeopardising the computational performance. The user can opt for either one of the models, using the same equation solver module.

For illustration, the explicit algebraic equation Yₛⁱ=αYₑⁱ could be replaced with the differential equation

$$Θ \frac{d {\hat{X}}^{i}}{dT} - {aY}_{e}^{i} + {\tilde{X}}^{i} = 0 ,$$

with the initial condition \tilde{X}ⁱ(t=0)=0, where θ is a time constant and \tilde{X}ⁱ a pseudo internal state variable. When t is much greater than the time constant, the solution \tilde{X}ⁱ(t) is constant, its derivative is therefore zero, and consequently αYₑⁱ=\tilde{X}ⁱ(t>>θ), For a sufficiently long time in comparison with the time constant, we thus end up with the solution of the explicit algebraic equation; all it needs is to set: Yₛⁱ=\tilde{X}ⁱ(t>>θ).

Hence, the operator could choose to describe an operation using a system of equations involving the internal state variables or an explicit algebraic equation. If at least one explicit algebraic equation is selected for at least one operation of the method, the system according to the invention replaces this explicit algebraic equation into a differential-algebraic equation in order to be able to integrate it into the system of equations of the entire process.

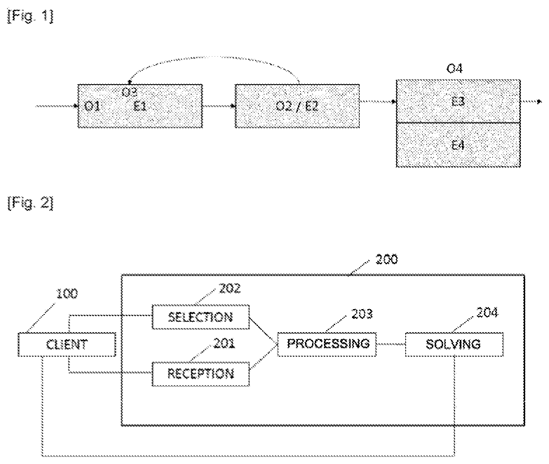

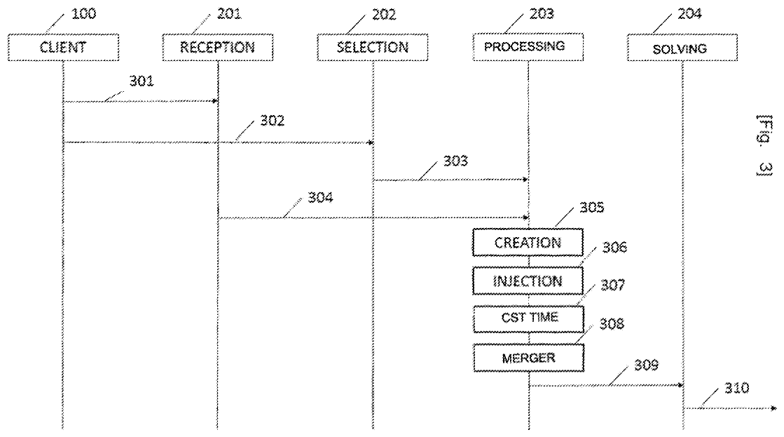

FIG. 2 illustrates a block diagram of a simulation module according to an embodiment of the invention. In the general context of the implementation of the system, a client device 100 connects to the simulation system 200 according to the invention. The simulation module 200 according to the invention comprises a reception module 201, optionally a selection module 202, a processing module 203 and a module for solving differential-algebraic equations 204.

The client device 100 connects to the reception module 201 in order to communicate to the reception module 201 an explicit algebraic equation representing a chemical or biochemical operation and relating a vector of input state variables representing initial chemical quantities of said operation to a vector of output state variables representing final chemical quantities of said operation.

The reception module 201 is connected to the processing module 203 in order to receive the explicit algebraic equation representing a chemical and biochemical operation and to create a differential-algebraic equation so that the steady-state solution of the algebraic equation differential thus created converges towards said vector of output state variables according to the explicit algebraic equation.

The processing module 203 is connected to the differential-algebraic equation solver module 204. Once the processing module 203 has performed the creation of the differential-algebraic equation, the solver module 204 solves the equation in order to obtain the vector of output state variables of the operation.

The differential-algebraic equation solver module 204 is connected to the client device in order to return the vector of output state variables of the operation.

According to one embodiment, the client device 100 communicates to the reception module 201 an explicit algebraic equation of the first operation and a differential-algebraic equation of the first operation. The client device 100 is connected to the selection module 202 to select a simulation via the explicit algebraic equation or a simulation via the differential-algebraic equation. Depending on the selected mode, the processing module creates a differential-algebraic equation converging towards said vector of output state variables according to the explicit algebraic equation or uses the existing differential-algebraic equation.

FIG. 3 illustrates the steps for performing the simulation in accordance with some embodiments.

The client module 100 communicates with the reception module 201 at step 301. During this step 301, the client module communicates to the reception module an explicit algebraic equation representing a first chemical or biochemical operation and relating a first vector of input state variables representing initial chemical quantities of said first operation to a first vector of output state variables representing final chemical quantities of said first operation. According to one embodiment, during this step 301, the client device could also communicate a differential-algebraic equation representing said first chemical or biochemical operation and relating a first vector of input state variables; and a first vector of internal state variables of said first operation as the unknown of the equation. According to one embodiment, during this step 301, the client device could also communicate a differential-algebraic equation representing a second chemical or biochemical operation and relating a second vector of input state variables; and a second vector of internal state variables of said first operation as an unknown of the equation.

According to one embodiment, the client module 100 communicates with the reception module at step 302. During this step 302, the client device selects a simulation mode. The simulation mode includes either the simulation of the chemical operation via the differential-algebraic equation or via the explicit algebraic equation.

During step 303, the selection module 202 communicates the selected simulation mode to the processing module and the reception module transmits 304 to the processing module the equation according to the selected simulation mode.

If a simulation from an explicit algebraic equation is selected for the first operation, the processing module performs the creating step 305 of a first differential-algebraic equation relating a first vector of input state variables; and a first vector of internal state variables of said first operation as the unknown of the equation. Next, the processing module injects 306 into the first differential-algebraic equation the expression of the vector of output state variables of said first operation according to said first explicit algebraic equation as a vector of pseudo internal state variables of said first operation, the steady-state solution of said differential-algebraic equation thus converging towards said first vector of output state variables according to the first explicit algebraic equation. Afterwards, the processing module sets the time constant at step 307. This time constant is less than the characteristic time of the first operation. This characteristic time could be provided by the user via the client module 100 and the reception module 201 to the processing module 203.

Afterwards, the processing module transmits the differential-algebraic equation thus obtained to the solver module for solving of the equation 309 in order to obtain the vector of output state variables of the first operation.

According to one embodiment, the solver module transmits this vector of output state variables of the first operation to the client device during a step 310.

According to an embodiment where the method comprises two operations including a second operation modelled from the start by a differential-algebraic equation, the processing module is configured to receive from the reception module the second differential-algebraic equation during a step 304 and to merge the second differential-algebraic equation with the first differential-algebraic equation during a step 308. Afterwards, the processing module transmits the system of differential-algebraic equations to the solver module during a step 309 which will solve this system and obtain a vector of output state variables of the process comprising the two operations.

According to an embodiment where an operation is represented by both an explicit algebraic equation and a differential-algebraic equation, the client device selects during step 302 a simulation mode. If the simulation via the explicit algebraic equation is selected, the processing module performs the steps of creation 305, injection 306 and setting of the time constant 307 then the step of merging 308 with the differential-algebraic equations of the other operations of the process. If the simulation via the differential-algebraic equation is selected, the processing module directly merges this differential-algebraic equation with the differential-algebraic equations of the other operations of the process. Thus, the present invention allows the combination of an accurate simulation using a differential-algebraic equation for some critical operations and the selection of a less accurate simulation using an explicit algebraic equation. The solution of this explicit algebraic equation being integrated into a differential-algebraic equation in order to enable the merger of the differential-algebraic equations in a single solver system.

The Simulation System

A simulation module as described hereinabove, and the corresponding method, could be implemented in a more global system and offering a complete industrial design tool enabling an entire design team to cooperate m the assessment and development of a chemical or biochemical process and the corresponding industrial installation.

This tool brings together various functionalities such as: data management (for example experimental data relating to chemical products, physico-chemical characteristics relating to raw materials or others), the processing of these data, the definition of operational units for carrying out a chemical process, the definition of basic operations entering into the chemical process, the definition of an installation for the implementation of the process with material equipment, the economic and financial assessment.

Where all these functionalities were, in the prior art, managed independently and inconsistently, they are grouped together in a system according to the invention and pooled in order to achieve the same goal. All these functionalities are implemented by modules and grouped together in a system while ensuring communication between the modules.

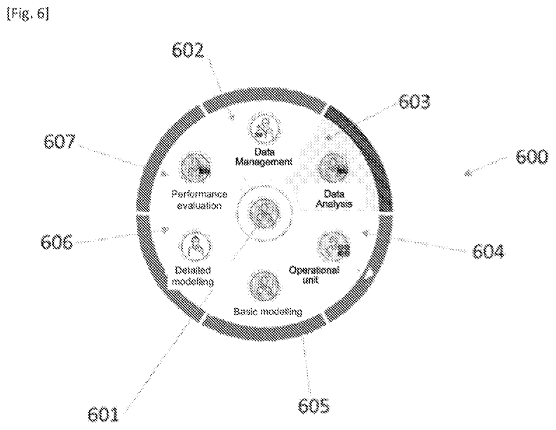

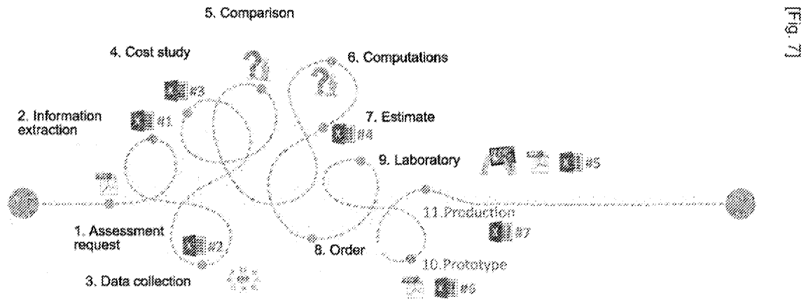

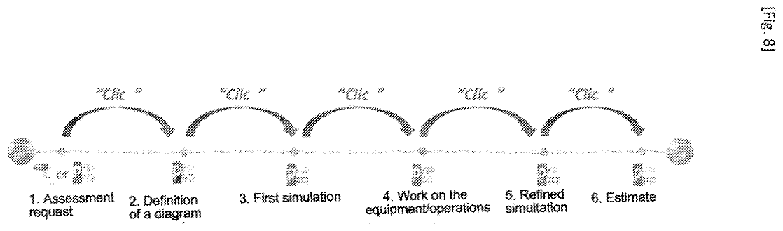

FIG. 6 schematically illustrates a system 600 according to some embodiments. It includes a central module 601 capable of controlling the system and coordinating the execution of the different modules. The system further includes modules 602 to 607 corresponding to the various functionalities listed hereinabove.

For example, each of the modules 604 to 606 relating to the definition of operational units, the definition of basic operations and of definition of a chemical installation could implement a simulation module 200 as described hereinabove. The client module 201 as described hereinabove could then be the central module 601. The module 602 allows storing and organising experimental data in a data structure that could be used by all of the other modules of the system. In particular, this module offers data relating to chemical species into which the raw materials, the reagents and the products will be decomposed throughout the process. The module 603 allows processing and analysing experimental data, for example carrying out statistical analyses, identifying parameters of a chemical process, etc. The module 607 allows carrying out performance estimates according to the experimental data derived from the module 602 or the module 603 or even results of simulations carried out by the modules 604 to 606. The module 607 also allows carrying out comparisons between different performance estimates or between a performance estimate and experimental data.

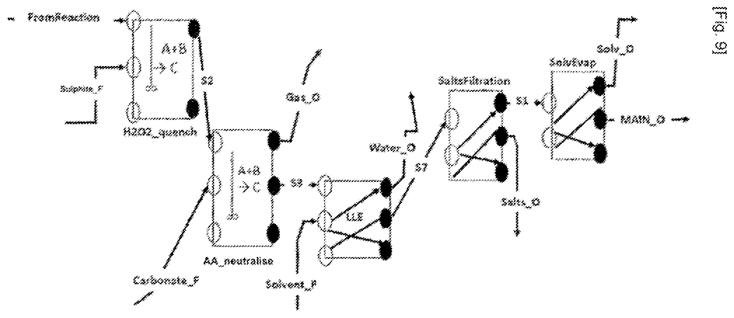

Such an architecture allows grouping together in a single tool information and functionalities that were scattered in the design modes of the prior art. Indeed, according to the prior art, as illustrated by FIG. 7, a lot of time and resources have been wasted in the collection and conversion of sparse data. Thus, when an assessment request has been received (1), it has been first necessary to extract the technical data (2) (nature of the process to be defined, raw materials, products, physico-chemical constraints, material constraints, cost constraints, etc.). Afterwards, it has been necessary to recover historical and know-how data enabling the assessment (3). This step already required the communication between different actors and could lead to a loss of information. Once the internal and historical data have been collected, it has been necessary to process them. Before launching the evaluation further, it has been necessary to carry out a cost study (4), involving new teams having their own tools and their own information. It was only afterwards that it was possible to start making comparisons (5), possibly, with development histories and finer computations (6) in terms of the process engineering. This new step still involved new teams with their own data and computation methods. Once the computations have been obtained, it has been then possible to proceed with a complete estimate (7) and to propose a final production cost. Once this has been communicated to a customer, for example, and once a firm order has been placed (8), the used data have been supplied to the laboratory responsible for carrying out the practical implementation of the defined and assessed process (9). A prototype has been then made (10) for a large-scale production launch (11).

The major problem encountered in the process of the prior art described hereinabove is that at each stage the participants have redefined the data and have carried out their processing independently of everything that could have been done before. This has led to a loss of efficiency and accuracy.

A system 600 according to the embodiments of the invention allows avoiding these difficulties by integrating the experimental data management into a single tool, prediction by simulation but also overall assessment by means of past experiences. This innovative approach, designated by GPX, an acronym for “Guess, Predict, Experimental”, allows carrying out relevant and rapid assessments.

The modules 605 to 606 participate to the “G” approach, which enables an overall assessment based on a simulation based on algebraic equations or past economic data. The modules 602 to 607 participate to the “P” approach by allowing an accurate simulation, using differential or differential-algebraic equations from experimental data. The modules 601, 602 and 607 participate to the “X” approach by providing experimental data and historical data to feed the other modules of the other approaches with relevant data.

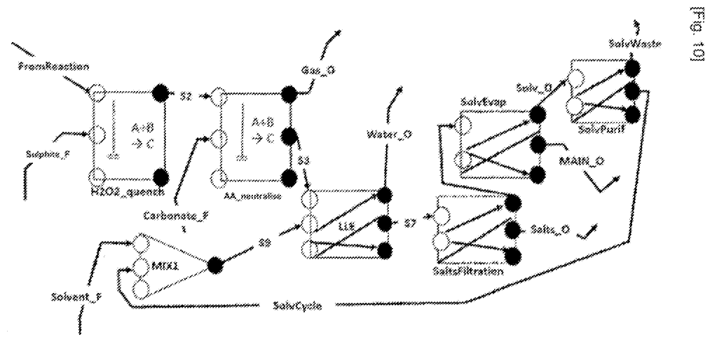

As illustrated by FIG. 8, a system according to embodiments thus allows carrying out, in the same data system and with consistent processing, all of the operations necessary for the assessment of a chemical process or of a corresponding installation: reception or definition of an assessment to be carried out with the corresponding technical data (1), definition of a block diagram with corresponding operations and equipment (2), a first simulation based on this diagram (3), a refinement of the pieces of equipment and of the operations (4), a more accurate simulation (5) and assessment (6).

Thus, a system according to some embodiments allows sharing information on an assessment project of a chemical process, capitalising on the information collected throughout the assessments and communicating the result of these assessments. All this is allowed without any loss of information and with great consistency in processing.

In general, the various modules of the system operate as described hereinafter.

The module 602 is used to organise the data that are shared in the system. In particular, it enables the recording of experimental scientific data concerning chemical species. The format in which these data are recorded is shared between all modules, allowing for an efficient and coordinated processing between the modules. In particular, the modules implementing simulations 604, 605 and 606 share this data format since they access the experimental data, in particular, as described hereinafter, the decomposition of the raw materials used by a chemical process. This is also the case of the module 607 which allows for comparisons, in particular between the simulation results and experimental data.

When launching a simulation, a project is defined within the system by means of a “project” file and a set of files: a first file describes the structure of the chemical or biochemical process, a file describes the sequence of the operations of the process and the last one represents the modelling of the process. Afterwards, different files are created to represent the simulations of each operational unit, basic operation and installation.

A particular characteristic of the system lies in the fact that in each file, it is possible to find the decomposition of the raw materials and the intermediate reagents decomposed according to chemical species whose characteristics are stored via the module 602.

Thus, according to the function of each module, write and read access will be different for each type of file. For example, only the central module 601 will be able to access the “write project” file. The other modules will only be able to access it in read mode for the purposes of executing their functionalities.

The module 602 will be the only one to write access the files representative of the experimental data. The other modules will only be able to access it in the read mode. As regards the module 603, it does not have write rights in the project description files. However, it has read access to the “project” file and to the files representing the experimental data. The modules capable of carrying out simulations 604, 605, 606 have access to the project file and to the files representing the experimental data. They could further access the files representing the simulations in the write mode.

Thus, for each type of file, only a single module is able to access it in the write mode. Conversely, different modules could share the reading of the same type of file (for example all modules could access the files representative of the experimental data. The sharing of information is thus facilitated in the system.

The project file could be in the form of an XML (extensible markup language) type file with three parts. A first part represents the list of the chemical species implemented in the project. Another part represents the list of raw materials with an identification and a composition with reference to the list of species. Thus, each raw material of the second part is decomposed into chemical species of the second part. A third part includes the other elements involved in the reactions such as catalysts, filter absorber resins, etc. with a respective identifier and some physico-chemical parameters.

The identifiers given to the chemical species, raw materials and others are valid for the project file and therefore for all the functions carried out by the different modules. Thus, all the modules could have access to the same decomposition and perform computations on this decomposition.

The chemical process is represented in the form of a block diagram, each block representing an operation or a facility. The blocks are related together according to the inputs and the outputs as illustrated in FIG. 1.

In the system, each block is represented by a set of three files.

A first file, of the XML type, includes two parts. A first part includes a list of flows with a respective identification and flow inputs and outputs. A second part includes process blocks with respective identifications, a position and lists of inputs and outputs corresponding to those of flows of the first part.

A second file describes the operations associated with the process. This second file, of the XML type, includes a first part with the input flows of the process (listed by their identification according to the first file) with the identification of the raw materials that they carry as well as their amount. It includes a second part with the lists of the process blocks and their operating temperatures.

The third file, also of the XML type, includes the modelling of the process with two parts. A first part with the input flows and the physical properties of each phase of the flow and the distribution of the species in each phase, A second part with the process blocks and the list of phases for each input flow with the physical properties and the distribution of the chemical species. If a chemical reaction takes place in the blocks, it is described in this part of the file.

This division into three files is identical for modelling by the modules 604, 605, 606.

Each process description type has a corresponding experimental data file. This file includes four parts. The first part includes a description similar to the one found in the first file describing the blocks and described hereinabove. The second part (which is optional) includes modelling data, similar to the ones of the third file describing the blocks and described hereinabove. The third part includes a description identical to that of the second file describing the blocks and described hereinabove. The fourth part is used to store measurement results.

The simulation files derived from the modules 604, 605, 606 are also of the XML type and include four parts. The first part is a copy of the “project” file. The second part is a copy of the first file describing the simulated block. The third part is a copy of the second file describing the simulated block. The fourth part is a copy of the third file describing the simulated block. An additional part may include a description of mass and energy balances expressed as a function of the flows for the mass and blocks for energy. As regards the files derived from the modules 604 and 605, they could also include mass balance and descriptions of the evolution of state variables such as temperature, flow rates, etc.

The assessments as processed by the module 607 are described in the system by a file that groups together all the data necessary for the computation of the costs and other performance assessment criteria. In general, this file may include flow identifiers in the reaction or the simulated installation which are consistent with those of the simulation files. It could also include identifiers of reference species that are consistent with the simulation files. Finally, the file may include unit costs for the raw materials used by the process or the simulated installation. These costs are entered for each raw material identified in a consistent manner with the simulation file.

In general, when an assessment corresponds to a raw material or a species, it is identified according to the identifiers of these raw materials or species. If it matches with a flow, it will be identified according to the identifier of the flow.

The system according to some embodiments processes the files as a transfer function that takes as arguments and as outputs some of the files described hereinabove. It is possible to process all these files in a consistent way by using as common data the species into which the raw materials are decomposed. Indeed, the experimental data generally deal with data relating to the species. The same applies to the numerical simulations. Finally, as regards to economic and financial data, these rather deal with the raw materials, but these are decomposed into species.

For example, the module 602 could take on as inputs the project file as well as other user data and produce the experimental data file therefrom. The use of the project file ensures that all the user data are expressed in terms of species versus raw materials. For example, again, the module 605 takes on as inputs the project file as well as user data (it could also use the experimental data file or others). It returns simulation files as output. Herein again, the definition by the project file in terms of chemical species allows returning results that are consistent with this representation. For example again, the module 607 takes on as input the simulation files created from the project file and outputs assessment files.

### EXAMPLES

In the following part, examples of use of a system according to the invention are described.

To generalise, the system will be implemented according to three main steps.

The first step 1 is a definition of the raw materials and of the species. It includes three substeps 1.1, 1.2, 1.3. It could be carried out using modules the 601 and 602.

The first substep 1.1 is a definition by the user of the raw materials entering the process. The second substep 1.2 is a decomposition of the raw materials into chemical species (for example: the raw material “azeotropic alcohol” is decomposed into its constituent chemical species, namely 96% ethanol and 4% water). The third substep 1.3 is an entry by the user of the chemical species or the biological materials produced in the process.

The second step is a definition of the Operation block diagram. It includes five substeps 2.1 to 2.5, It could be carried out by the module 605.

In the first substep 2.1, an operation block diagram consisting of operation blocks is created by a user or from a laboratory recipe. A library BBOP of operation blocks BOP is provided, each operation block having a number of inputs and outputs, in species, raw materials, or energy and representing an elementary operation, such as a mixing, a reaction, a separation, a heating. These operation blocks BOP represent transformations of the flows or amounts regardless of the volume or time required thereby. These operation blocks BOP are not associated with equipment and do not contain any notion of productivity.

In the second step 2.2, there is a consideration by the system of the relationships between the inputs and the outputs of each of the selected operation blocks BOP. The user can provide these relationships by extracting a model from a first library of operation models MOP1 and possibly by specifying parameters of this model, for example a ratio between an input and output flow rate of the block. These relationships could relate to various parameters such as temperatures, pressures, material amounts or flows, concentrations in different phases. At this stage, this consists of a so-called “Guess” mode: only the intuition, the experience of the user are used, no physico-chemical information is necessary.

In step 2.3, by means of the operation block diagram and the relationships between the inputs and the outputs of each of the selected Operation blocks BOP, the system determines overall balances of the process. These overall balances may concern the production of the chemical species or biological materials produced in the process, the consumption of raw materials or energy. This determination could be carried out either continuously (flow data) or discontinuously (amount of data per batch). At this stage, the system could also determine a first estimate of the performance criteria of the process based on the consumptions of raw materials and/or energy, this criterion possibly being an economic or environmental one.

In step 2.4, the possibility is then offered to the user to repeat step 2.1, by selecting other operation blocks or by arranging them otherwise to obtain another operation block diagram a repeating step 2.2 while choosing other relationships between the inputs and the outputs of the selected operation blocks, then performing step 2.3 again for determining the overall balances, and continuing until obtainment of satisfactory overall balances.

Optionally, in step 2.5, the possibility is offered to the user upon completion of a step 2.3 or 2.4 to replace the relationships between the inputs and the outputs of some of the operation blocks BOP with relationships extracted from a second library of operation models MOP2. This second library of operation models MOP2 includes more elaborate characteristics, taking into account, for example, thermodynamic information, or phase composition. The system then determines the overall balances, following step 2.3, based on the improved characteristics. The user will advantageously select the operation blocks for which the input/output relationships should be replaced among the most critical operation blocks in the process. At this stage, the system could also propose a second determination of the performance criteria based on the consumption of raw materials and/or energy.

The third step is the definition of equipment diagrams. It includes six substeps 3.1 to 3.6. It could be carried out by the modules 604, 606.

In the first substep 3.1, there is a transformation of the operation block diagram into an equipment diagram representing the industrial installation to be designed. Equipment is chosen from a library of generic equipment BEQGEN or from a library of specific equipment BEQSPEC to carry out the operations of one or several operation block(s) BOP of the operation block diagram, until the transformation of all of the operation blocks of the operation block diagram. The pieces of equipment of the BEQGEN library are generic pieces of equipment: their characteristics could remain rather vague, even idealised: a reactor may for example be defined as a perfectly mixed adiabatic system, independently of the means to achieve this result. The pieces of equipment of the BEQSPEC library are specific pieces of equipment. They could then have very specific characteristics enables the direct creation of an equipment diagram without prior creation of an operation diagram. Such a shortcut may for example be useful if it is desired to represent an already existing installation.

In a step 3.2, there is a specification of the different operations carried out in each piece of equipment. For each operation, its nature is specified in particular (loading of raw material, reaction, emptying, temperature change, etc.) as well as its duration. All of these equipment specifications correspond to an operating procedure like those used in industrial workshops. This operating procedure does not involve any modelling elements.

Step 3.3 is a determination by the system of operating parameters of the industrial installation represented by the equipment diagram using the model libraries MOP1 and MEQ1 as well as the connectivity between the pieces of equipment. The models of the library MEQ1 are intended to describe the operations performed in the pieces of equipment as entered at step 3.2. As with the library MOP1, these are models based on a macroscopic description of the phenomena and not involving any physico-chemical data. The operating parameters of the industrial installation may include productivities, yields, product quality parameters, such as purity, waste production, energy consumption. At this stage, the system could determine a first evaluation of a performance criterion based on the consumption of raw materials, energy, the size and operating mode of the equipment. For example, this criterion could be an economic or environmental one.

At step 3.4, the possibility is then offered to the user to repeat step 3.1, by selecting other pieces of equipment, and/or to repeat step 3.2 while choosing other characteristics for the operations carried out in the pieces of equipment, then performing step 3.3 again for determining operating parameters, and continuing until obtaining parameters complying with the set constraints.

Optionally, at step 3.5, the possibility is offered to the user upon completion of a step 3.3 or 3.4 to replace the characteristics of some operations carried out in generic or specific pieces of equipment of the equipment diagram with characteristics extracted from a second library of models of operations MEQ2 performed in pieces of equipment. The models derived from this second library are much more elaborate and allow, for example, computing conversions in reactors or separation performance in distillation from physicochemical data. The system then determines again the operating parameters of the industrial installation; according to step 3.3, based on the improved characteristics. At this stage, the system could determine performance criteria based on the consumption of raw materials, energy, the size and the operating mode of the pieces of equipment.

Optionally, at step 3.6, the possibility is offered to the user upon completion of a step 3.3 or 3.4 to replace the characteristics of some operations performed in generic or specific pieces of equipment of the equipment diagram with characteristics extracted from a third library MEQ3 of models of operations performed in pieces of equipment. The models from this third library are detailed models and allow, for example, the representation of non-idealities of mixing or complex hydrodynamic phenomena by taking into account some geometric characteristic of specified pieces of equipment, Again, the system then determines operating parameters of the industrial installation, according to step 3.3, based on the improved characteristics. At this stage, the system can determine performance criteria based on the consumption of raw materials, energy, the size and the operating mode of the pieces of equipment. This substep could be carried out by the module 607.

### Example 1

Two examples of use to simulate a project will now be described.

A first example of application of this simulation method is given hereinafter. It is inspired by a real case and concerns a purification located downstream of an organic synthesis that produces delta-valerolactone. The objective is to minimise the production cost of delta-valerolactone. This objective could be broken down into the following two questions: is it possible to propose simple improvements to the current purification technology? Is it possible to offer other purification technologies with lower costs?

As disclosed in the section hereinbelow, the initial information is limited, which prevents any recourse to elaborate models (whether in terms of thermodynamics or kinetics) unless resorting to long and costly measurement campaigns. The top-down approach disclosed herein consists in carrying out a first series of computations based on the immediately available or accessible free information. Then, based on these computations, the additional information that is really needed is identified in order to minimise the effort of collecting this information.

At the end of the reaction, the delta-valerolactone is in an aqueous solution which also contains cyclopentanone, hydrogen peroxide and acetic acid. It is sought to remove these last two species and to dehydrate the product. The conventional procedure consists in: neutralising the peroxide with sodium sulphite, neutralising the acetic acid with sodium carbonate, extracting the organic molecules using an organic solvent, evaporating the water to precipitate the salts, filtering the salts, evaporating the organic solvent. We will also consider a setup based on thermal neutralisation of peroxide and removal of acetic acid by distillation.

The first step is the definition of the raw materials and of the species.

The chemical species involved in the processes studied are listed in Table 2. Some of them originate from the raw materials listed in Table 1, others are generated by chemical reaction.

For each of the species in Table 2, some information that anybody could find for free and quickly is provided.

TABLE 1
raw materials and species injected into the DVL purification processes

| Raw materials | Associated species (weight %) |
|---|---|
| Reaction mixture | 54.01% H2O; 1.11% CP; 3.48% H2O2; |
| | 16.27% AcH; 25.13% DVL |
| Fresh MIBK | 100% MIBK |
| 20% sodium sulphite | 80% H2O; 20% Na2SO3 |
| 20% sodium carbonate | 80% H2O; 20% Na2SO3 |
| Namely 4 raw materials | Namely 8 associated species |

TABLE 2
list of species involved in the DVL purification processes

| | | Name | Molar | Normal |
|---|---|---|---|---|
| Common name | | in this | mass | boiling |
| or formula | CAS | document | (g/mol) | point (° C.) |
| cyclopentanone | 120-92-3 | CP | 84.12 | 131 |
| delta- | 542-28-9 | DVL | 100.12 | 230 |
| valerolactone | | | | |
| Water | 7732-18-5 | H2O | 18.02 | 100 |
| H₂O₂ | 7722-84-1 | H2O2 | 34.01 | — |
| Acetic acid | 64-19-7 | AcH | 60.05 | 118 |
| Sodium | 7757-83-7 | Na2SO3 | 126.04 | — |
| sulphite | | | | |
| Sodium | 497-19-8 | Na2CO3 | 105.99 | — |
| carbonate | | | | |
| CO₂ | 124-38-9 | CO2 | 44.01 | — |
| Sodium acetate | 127-09-3 | AcNa | 82.03 | — |
| Sodium sulphate | 7757-82-6 | Na2SO4 | 142.04 | — |
| MIBK | 108-10-1 | MIBK | 100.16 | 116 |
| Dioxygen | 7782-44-7 | O2 | 32.00 | — |

The second step is the definition of operation block diagrams.

In the approach described hereinabove, the process modelling step 2.1 consists in the development of an operation block diagram. Each icon in this diagram represents an operation (i.e., an action on a flow or on an amount). At step 2.2, these operations are described by the relationships between their inputs and their outputs and could be represented by very simple empirical models or by more accurate thermodynamic models, At step 23, solving the balance equations associated with the relationships between the inputs and the outputs of each block and the connectivity of the blocks together will enable us to know the mass and heat flows (or amounts) at each location of the system, and in particular at the output.

The considered three operation diagrams are represented:

In FIG. 9: conventional scheme of purification by saline neutralisation and liquid-liquid extraction without solvent recycling (case 1)

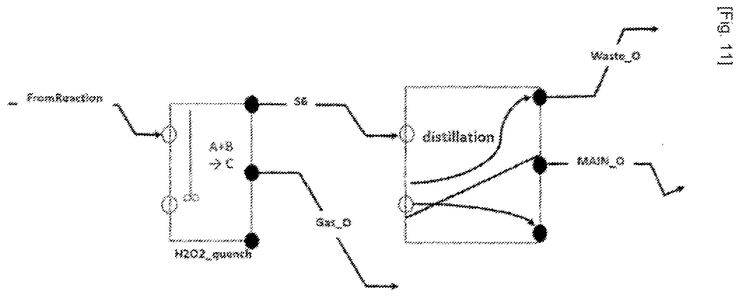

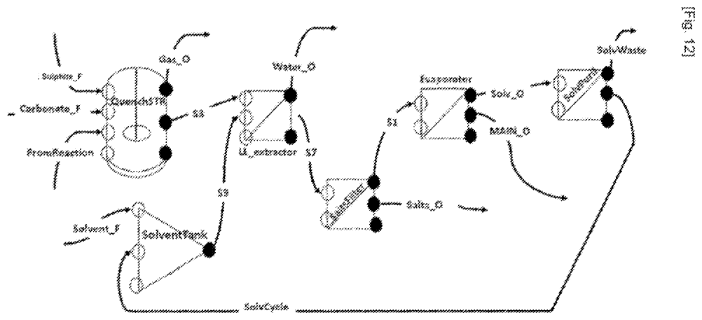

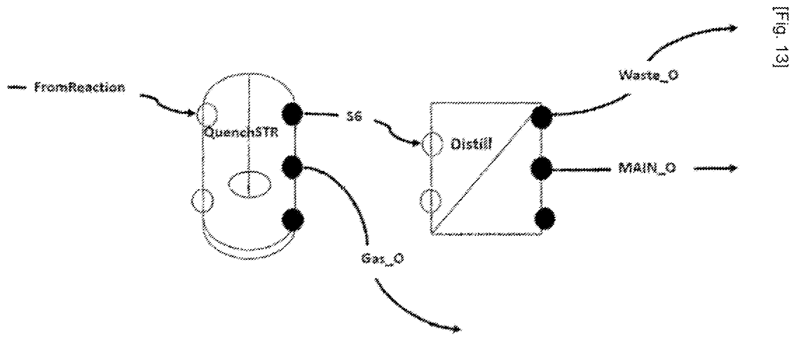

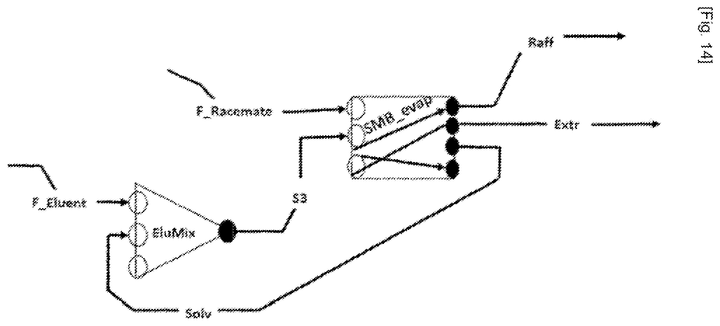

In FIG. 10: alternative scheme of purification by saline neutralisation and liquid-liquid extraction with solvent recycling (case 2) In FIG. 11: alternative scheme of purification by thermal neutralisation and distillation (case 3)

The operation block diagrams could be read in flow (kg/h) for continuous processes as well as in treated amount (kg) per operation for discontinuous processes.

In step 2.2, relationships between the inputs and outputs of the operation blocks are defined.

The models of the MORI library propose a very macroscopic description of the effect of each operation block on the through-flows or through amounts. This macroscopic description is compatible with the use of assumptions based on intuition and experience.

The diagram of FIG. 9 includes two reactions H2O2_quench and AA_neutralise corresponding respectively to the neutralisation of H2O2 by Na2SO3 and of AcH by Na2CO3. This scheme also includes three separations LLE, SaltsFiltration and SolvEvap corresponding respectively to the extraction of DVL and CP by an organic solvent (with removal of water and precipitation of the salts), filtration of the salts, evaporation of the solvent.

The diagram of FIG. 10 is extremely similar to that of FIG. 1 with the addition of the operation SolvPurif— intended to separate the solvent to be discarded from that which could be recycled—as well as the operation MIX1 intended to mix the fresh solvent and the recycled solvent.

The diagram of FIG. 11 simply includes the reaction block H2O2_quench corresponding to the thermal neutralisation of H2O2 and the separation block distillation corresponding to the removal of H2O and AcH.

In general, the mixing operations simply consist in grouping together the masses (or mass flow rates) of each species contained in ail of the input flows. As regards the separation and reaction operations with several output flows, a ratio is provided by the user for each species in order to indicate its distribution between the output flows. In the examples hereinafter, these ratios are often 0% or 100% but they could take on any value between 0% and 100%. Moreover, this system of ratios is herein illustrated on situations with two output flows, but it also applies to situations where these flows are more numerous as well as to balances between phases.

The reactive phenomena represented by the reaction blocks are described in Table 3

TABLE 3
reaction models

| Name | Equation | Conversion |
|---|---|---|
| H2O2_quench (cases 1 | H2O2 + Na2SO3 → | 100% of H2O2 |
| and 2) | H2O + Na2SO4 | |
| AA_neutralise | 2 AcH + Na2CO3 → | 100% of AcH |
| | 2 AcNa + H2O + CO2 | |
| H2O2_quench (case 3) | 2 H2O2 → 2 H2O + O2 | 100% of H2O2 |

The table hereinbelow includes partition ratios corresponding to the assumption according to which, during the chemical neutralisation of AcH, all of the generated CO2 is evacuated without liquid entrainment. In Table 9, an identical assumption is made for the release of O2 in the case of a thermal neutralisation.

TABLE 4
partition ratios of the species between the outputs of AA_neutralise

| | Gas_O | S3 |
|---|---|---|
| CO2 | 100% | 0% |
| Other species | 0% | 100% |

The ratios of the table hereinbelow represent a liquid-liquid extraction where the aqueous and organic phases are completely immiscible. For the removal of water, the entire load of the operation is brought to 100° C., water is thus evaporated (state change enthalpy of 2260 id/kg). Then, all of the output flows are brought back to 25° C. in the liquid state.

TABLE 5
partition ratios of the species between the outputs of LLE

| | Water_O | S7 |
|---|---|---|
| H2O, CO2 | 100% | 0% |
| Other species | 0% | 100% |

As regards the removal of salts (cases 1 and 2), filtration is assumed without retention of liquid in the retained solid.

TABLE 6
partition ratios of the species between the outputs of SaltsFiltration

| | Salts_O | S1 |
|---|---|---|
| Na2SO3, Na2CO3, AcNa, Na2SO4 (solids) | 100% | 0% |
| Other species | 0% | 100% |

As regards the operation SolvEvap, it is considered that the entire load is brought to 120° C. and that MIBK is thus evaporated then the two output flows are brought back to 25° C. in the liquid state. The state change enthalpy is considered as equal to 400 kJ/kg,

TABLE 7
partition ratios of the species between the outputs of SolvEvap

| | Water_O | MAIN_O |
|---|---|---|
| MIBK | 100% | 0% |
| CP, DVL | 0% | 100% |
| Other species | 50% | 50% |

in the process of FIG. 10, it is considered that part of the solvent cannot be reused and that the recycling concerns 95% of the solvent (cf, Table 8). As for SolvEvap, an evaporation at 120° C. of the MIBK flow is considered.

TABLE 8
partition ratios of the species between the outputs of SolvPurif

| | SolvWaste | SolvCycle |
|---|---|---|
| MIBK | 5% | 95% |
| Other species | 100% | 0% |

TABLE 9
partition ratios of the species between the outputs of
H2O2_quench in the case of thermal neutralisation

| | Gas_O | S6 |
|---|---|---|
| O2 | 100% | 0% |
| Other species | 0% | 100% |

TABLE 10
partition ratios of the species between the outputs of distillation

| | Waste_O | MAIN_O |
|---|---|---|
| O2, H2O, AcH | 100% | 0% |
| CP, CVL, AcNa | 0% | 100% |
| H2O2 | 0% | 100% |

In this case, the system of partition ratios is used to account for generally ideal separations. By its mere structure, this system allows keeping aware of this assumption of ideality and would allow representing deviations from ideality, for example by considering ratios of 97%/3% instead of the current 100%/0%.

Afterwards, the overall balances are determined at step 2.3.

In the three configurations, the purpose is to treat a load of 4050 kg of reaction mixture (cf. composition in Table 1).

The sulphite and carbonate flow rates are computed so as to completely neutralise H2O2 and AcH. The flow rate of fresh MIBK is computed so that in LLE. 4000 kg of MK are mixed with the load of DVL and CP.

In the basic case, the material balance of Table 11 and the energy balance of Table 12 are obtained.

TABLE 11
description of the flows of the process in the basic case (case 1)

| | Flow | Raw materials | Amounts |
|---|---|---|---|
| OUTPUTS | MAIN_O | | 1,063 kg |
| | Gas_O | | 242 kg |
| | Water_O | | 6,921 kg |
| | Salts_O | | 1,525 kg |
| | Solv_O | | 4,000 kg |
| INPUTS | FromReaction | Reaction mixture | 4,050 kg |
| | Sulphite_F | 20% Sodium | 2,700 kg |
| | | sulphite | |
| | Carbonate_F | 20% Sodium | 3,000 kg |
| | | carbonate | |
| | Solvent_F | Fresh MIBK | 4,000 kg |
| INTERNALS | S2 | | 6,750 kg |
| | S3 | | 9,509 kg |
| | S7 | | 6,599 kg |
| | S1 | | 5,063 kg |

TABLE 12
heat exchanges in the basic case (case 1)

| Operation block | Direction of the exchange | Energy (kWh) |
|---|---|---|
| H2O2_quench | Input to the process | 259 |
| AA_neutralise | Input to the process | 68 |
| LLE | Input to the process | 4,808 |
| | Output from the process | 5,197 |
| SolvEvap | Input to the process | 678 |
| | Output from the process | 678 |

In the case with recycling of the extraction solvent, the results of Table 13 and Table 14 are obtained. Compared to the basic case, the main differences occur at the level of the solvent inlets and outlets. Recycling allows reducing them considerably by means of an additional input of energy.

TABLE 13
description of the flows of the process in the case with
recycling of the extraction solvent (case 2)

| | Flow | Raw materials | Amounts |
|---|---|---|---|
| OUTPUTS | MAIN_O | | 1,063 kg |
| | Gas_O | | 242 kg |
| | Water_O | | 6,921 kg |
| | Salts_O | | 1,525 kg |
| | SolvWaste | | 200 kg |
| INPUTS | FromReaction | Reaction mixture | 4,050 kg |
| | Sulphite_F | 20% Sodium | 2,700 kg |
| | | sulphite | |
| | Carbonate_F | 20% Sodium | 3,000 kg |
| | | carbonate | |
| | Solvent_F | Fresh MIBK | 200 kg |
| INTERNALS | S2 | | 6,750 kg |
| | S3 | | 9,509 kg |
| | S7 | | 6,599 kg |
| | S1 | | 5,063 kg |
| | Solv_O | | 4,000 kg |
| | SolvCycle | | 3,800 kg |
| | S9 | | 4,000 kg |

TABLE 14
heat exchanges in the case of the process with recycling of the
extraction solvent (case 2)

| Operation block | Direction of the exchange | Energy (kWh) |
|---|---|---|
| H2O2_quench | Input to the process | 259 |
| AA_neutralise | Input to the process | 68 |
| LLE | Input to the process | 4,808 |
| | Output from the process | 5,197 |
| SolvEvap | Input to the process | 678 |
| | Output from the process | 678 |
| SolvPurif | Input to the process | 600 |
| | Output from the process | 600 |

In the case of the process with thermal neutralisation of peroxide, the material balance of Table 15 and the energy balance of Table 16 are obtained. The amounts of material and energy are substantially lower than those of the other configurations. Indeed, neither extraction solvent nor water (with sulphite and carbonate) is added.

TABLE 15
description of the flows of the process in the case of thermal
neutralisation of the peroxide (case 3)

| | Flow | Raw materials | Amounts |
|---|---|---|---|
| OUTPUTS | MAIN_O | | 1,063 kg |
| | Gas_O | | 66 kg |
| | Waste_O | | 2,921 kg |
| INPUTS | FromReaction | Reaction mixture | 4,050 kg |
| INTERNALS | S6 | | 3,984 kg |

TABLE 16
heat exchanges in the case of thermal neutralisation of the
peroxide (case 3)

| Operation block | Direction of the exchange | Energy (kWh) |
|---|---|---|
| H2Q2_quench | Input to the process | 315 |
| distillation | Input to the process | 1,883 |
| | Output from the process | 2,157 |

These six tables form three simulation results.

For step 2.4, when a laboratory recipe is available—i.e., an experimental procedure possibly accompanied by result elements—the system covered by this invention enables the entry of the information present in the recipe. It is possible to enter all the information of the recipe without prejudging its possible later use; at the same time, no information has imperatively to be provided except the nature of each operation. Once this recipe has been entered, the computer program is capable of interpreting its content to create the structure of an operation block diagram based on this content.

The following lab recipe could be used as a basis for creating the flowchart of FIG. 9.

Add 4.05 kg of reaction mixture and 2.75 kg of 20% sodium sulphite. Leave to react for 2 hours Add 3.5 kg of 20% sodium carbonate, leave to react for 1 hour. An off-gassing is observed

Add 4 kg of fresh MIBK. Mix and let settle. Evaporate the aqueous phase at 100° C. A precipitation is observed.

Filter the mixture Concentrate by evaporation of the solvent at 120° C.

A laboratory recipe may for example be derived from a scientific article relating to a synthesis experiment. This article then forming one of the first sources of information on the approach.

For the assessment of the costs of step 2.5, the balances of the previous six tables will now serve as a basis for economic computations, they allow computing the variable part of the production cost. These costs will be reported per kilogram of DVL in the flow MAIN_O.

The used assumptions are disclosed hereinbelow. For many, these are orders of magnitude derived from experience.

TABLE 17
unit cost of the raw materials

| Raw material | Price (€/kg) |
|---|---|
| Reaction mixture | 7.00 |
| 20% Sodium sulphite | 0.38 |
| 20% Sodium carbonate | 0.25 |
| Fresh MIBK | 0.98 |

TABLE 18
processing cost of the material secondary outputs

| Flow | Treatment cost (€/kg) |
|---|---|
| Gas_O (all cases) | 0.00 |
| Water_O (cases 1 and 2) | 0.15 |
| Salts_O (cases 1 and 2) | 0.10 |
| Solv_O (case 1) | 0.15 |
| SolvWaste (case 2) | 0.15 |
| Waste_O (case 3) | 0.15 |

A cost of 0.10 €/kWh is assigned to the energy flows brought to the processes (heating/evaporation). A cost of 0.05 €/kWh is assigned to the energy flows drawn from the processes (cooling/condensation). The material balances relate to species, the economy relates to the value of the raw materials, hence the importance of step 1.2.

Based on the material and energy balances as well as the economic assumptions hereinabove, we end up with the variable costs of the three versions of the process as disclosed in Table 19.

TABLE 19
contribution of each input to the variable part of the production
cost of DVL. The values are expressed in €/kg per kg of DVL
in the flow MAIN_0

| | Process 1 | Process 2 | |
|---|---|---|---|
| | Basic case | Recycling of | Process 3 |
| | without | the extraction | Thermal |
| | recycling | solvent | neutralisation |
| Reaction mixture | 27.86 | 27.86 | 27.86 |
| 20% sodium sulphite | 1.01 | 1.01 | — |
| 20% sodium carbonate | 0.74 | 0.74 | — |
| Fresh MIBK | 3.85 | 0.19 | — |
| Treatment of the effluents | 1.76 | 1.20 | 0.43 |
| Energy | 0.86 | [01761] 0.95 | 0.32 |
| TOTAL | 36.07 | 31.94 | 28.61 |
| Deviation with respect to | — | −11.4% | −20.7% |
| the basic case | | | |

It could be noticed that, thanks to the recycling of solvent, the process 2 allows for interesting savings both at the level of the fresh MIBK and of the treatment of effluents. The counterpart in terms of energy is minimal.

The process 3 allows for even greater savings because the used neutralisation technique significantly reduces the amounts of raw materials to be infected and the amounts of solvents (including water) to be separated and treated again.

When comparing the processes 1 and 2, an obvious difference in variable costs can be noticed. Yet, knowing that the two configurations are extremely close, it is possible to assume that the device enabling recycling will not induce additional fixed costs that are significant enough to offset the advantage of the process 2 in terms of variable costs. Hence, it is possible to consider that the process 1 will necessarily be less competitive than the process 2. Therefore, it will no longer be considered for the rest of the study.

When comparing the processes 2 and 3, it could be noticed that the process 3 is obviously more advantageous in terms of variable costs. Nonetheless, considering the fundamental differences (nature of the chemical reactions, structure of the process, etc.) between these two processes, it seems necessary, to draw a serious conclusion, to take into account the fixed costs and, through these, the notions of time and equipment.

In step 3.1, each of the operation blocks of two preserved diagrams is assigned to a piece of equipment. Note that several operations could be carried out in the same piece of equipment (for example the two neutralisation reactions in the case of the process 2).

The system has a library of generic equipment BEQGEN and a library of specific equipment BEQSPEC, with each piece of equipment having its own characteristics.

In the present case (cf. Table 20 and Table 21), all of the considered equipment is derived from the BEQGEN library. In the case of reactors, a first technological choice is made since it is decided to use stirred reactors, a very classic choice in the case of discontinuous processes. In the case of separators, generic separators are currently used. Thus, it is not necessary to take a position from the outset on questions such as the nature of the filter or the number of trays of the distillation column. Nonetheless, we are aware that the choice made currently limits the accuracy of the results relating to the separation equipment.

A first (somehow arbitrary) volume value is given for each piece of equips ent. This could be modified later on.

TABLE 20
assignment of the operations to equipment in the case of the process 2
(recycling of the extraction solvent)

| | Equipment | | |
|---|---|---|---|
| Operation block | Name | Type | Volume (m³) |
| H2O2_quench | QuenchSTR | Stirred reactor | 14 |
| AA_neutralise | | | |
| LLE | LL_extractor | Generic separator | 18 |
| SaltsFiltration | SaltsFilter | Generic separator | 10 |
| SolvEvap | Evaporator | Generic separator | 8 |
| SolvPurif | SolvPurif | Generic separator | 6 |
| MIX1 | SolventTank | Vat | 6 |

TABLE 21
assignment of the operations to equipment in the case of the
process 3 (thermal neutralisation)

| | Equipment | | |
|---|---|---|---|
| Operation block | Name | Type | Volume (m³) |
| H2O2_quench | QuenchSTR | Stirred reactor | 6 |
| distillation | Distill | Generic separator | 6 |

As regards step 3.2, it could be noticed that at the start of the process 2 (liquid-liquid extraction with solvent recycling), the equipment SolventTank contains 3800 kg of MIBK. Afterwards, the applied operating procedure is disclosed in Table 22. For several operations, an arbitrary duration of 0.01 h is applied because it is assumed that the duration of these steps is negligible compared to the durations of reactions and separations.

TABLE 22
operating procedure of the process 2 (liquid-liquid extraction with
recycling of the solvent)

| | | Duration |
|---|---|---|
| Rank | Operation | (h) |
| 1 | Loading of 4050 kg of Reaction mixture in | 0.01 h |
| | QuenchSTR | |
| 2 | Loading of 2750 kg of 20% Sodium sulphite in | 0.01 h |
| | QuenchSTR | |
| 3 | Reaction in QuenchSTR | 2 h |
| 4 | Loading of 3500 kg of 20% Sodium carbonate in | 0.01 h |
| | QuenchSTR | |
| 5 | Reaction in QuenchSTR | 1 h |
| 6 | Partial emptying of QuenchSTR by Gas_O | 0.01 h |
| 7 | Total emptying of QuenchSTR in LL_extractor | 0.01 h |
| 8 | Loading of 200 kg of Fresh MIBK in SolventTank | 0.01 h |
| 9 | Total emptying of SolventTank in LL_extractor | 0.01 h |
| 10 | Stirring in LL_extractor | 4 h |
| 11 | Partial emptying of LL_extractor by Water_O | 1 h |
| 12 | Total emptying of extractor in SaltsFilter | 0.01 h |
| 13 | Partial emptying of SaltsFilter in Evaporator | 2 h |
| 14 | Total emptying of SaltsFilter by Salts_O | 0.01 h |
| 15 | Partial emptying of Evaporator in SolvPurif | 2 h |
| 16 | Partial emptying of SolvPurif in SolventTank | 2 h |
| 17 | Total emptying of Evaporator by MAIN_O | 0.01 h |
| 18 | Total emptying of SolvPurif by SolvWaste | 0.01 h |

As regards the process 3 (thermal neutralisation), the operating procedure is disclosed in Table 30.

TABLE 23
operating procedure of the process 2 (liquid-liquid extraction with
recycling of the solvent)

| | | Duration |
|---|---|---|
| Rank | Operation | (h) |
| 1 | Loading of 4050 kg of Reaction mixture in | 0.01 h |
| | QuenchSTR | |
| 2 | Heating of QuenchSTR to 80° | 2 h |
| 3 | Reaction in QuenchSTR | 22 h |
| 4 | Partial emptying of QuenchSTR by Gas_O | 0.01 h |
| 5 | Total emptying of QuenchSTR in Distill | 0.01 h |
| 6 | Total emptying of Distill by Waste_O and MAIN_O | 5 h |

At step 3.3, the system determines operating parameters according to guess models (MEQ1)

The models of the MEQ1 library are intended to describe the operations taking place in the equipment (cf. Table 22 and Table 23). They are extremely similar in their principle and structure to those of the MOP1 library.

As regards the reaction operations (the operations 3 and 5 for the process 2, the operation 3 for the process 3), the used models are strictly identical to those of Table 3.

As regards the operation 6 of the process 3 (the distillation of water and acetic acid), the model described in Table 10 is used identically. The other separations represented on the operation diagram of the process 2 are transcribed in the form of binomials (partial emptying+total emptying). As regards the partial emptying, the partition ratios between the extracted material and the material remaining in the equipment are those of Tables 4 to 8. This actually amounts to keeping these ratios for the distribution of the species between the output flows.

Hence, the switch from the operation diagram into the equipment diagram does not require a lot of additional information as long as equipment from the BEQGEN library and operation models from the MEQ1 library are used. The additional information essentially consists in assigning durations to the operations.

In both cases, the input and output material and energy balances obtained by the computation are exactly identical to those obtained with the operation diagrams (cf. Tables 13 to 16).

The computation also allows obtaining the filling rate of each piece of equipment likely to store content as well as the total time of the operating sequence (cf. Table 24 and Table 25). In the case of the process 2, it could be noted that some initially specified volumes have to be changed in order to achieve reasonable occupancy rates. The methodology disclosed herein allows for such an iterative process.

TABLE 24
result elements for the simulation of the equipment
diagram of the process 2

| | | Volume | |
|---|---|---|---|
| Data | Value | Old | New |
| Occupancy rate of QuenchSTR | 67% | 14 m³ | 12 m³ |
| Occupancy rate of LL_extractor | 94% | 18 m³ | 22 m³ |
| Occupancy rate of Salt/Filter | 82% | 10 m³ | 10 m³ |
| Occupancy rate of Evaporator | 93% | 8 m³ | 10 m³ |
| Occupancy rate of SolvPurif | 102% | 6 m³ | 8 m³ |
| Occupancy rate of SolventTank | 97% | 6 m³ | 8 m³ |
| Total time | 14.1 h | | |

TABLE 25
result elements for the simulation of the equipment diagram
of the process 3

| Data | Value |
|---|---|
| Occupancy rate of QuenchSTR | 67.5% |
| Occupancy rate of Distill | 66.4% |
| Total time | 29.03 h |

At this stage, we have the necessary information to proceed with a computation of fixed costs.

The assumptions relating to the variable costs remain valid. Those relating to the fixed costs are disclosed hereinbelow.

To calculate the investment cost of a piece of equipment, reference is made to a size and to a reference price according to the following formula:

$$Price of the equipment ( k ) = Reference price ( k ) {( \frac{size}{Reference size} )}^{elasticity} \qquad ( 1 )$$

The detail of the CAPEX of the two studied processes appears in Table 26 and Table 27.

The cost of the equipment is considered to be amortised over 64,000 hours. We also consider a maintenance cost of 5% of the CAPEX per year of 8,000 hours.

In both cases, a process downtime of 2.4 hours is considered for each cycle. When the process is in operation, it is considered that each piece of equipment (except the solvent storage) mobilises a full-time equivalent costing 1,000 [symbol] /day.

A quality control cost of 100,000 €/year and an overhead cost of 25% of the other fixed costs are considered.

TABLE 26
size, economic parameters and cost of each equipment for the process 2

| | | | | | Cost (k€) |
|---|---|---|---|---|---|
| | | Ref. | Ref. price | | of the |
| Equipment | Size | size | (k€) | Elasticity | equipment |
| QuenchSTR | 12 m³ | 3 m³ | 1,000 | 0.6 | 2,297 |
| LL_extractor | 22 m³ | 3 m³ | 1,500 | | 4,958 |
| SaltsFilter | 10 m³ | | | | 3,089 |
| Evaporator | 10 m³ | | | | 3,089 |
| SolvPurif | 8 m³ | | | | 2,702 |
| SolventTank | 8 m³ | 3 m³ | 100 | | 180 |

| | |
|---|---|
| Total CAPEX | 16,815 |

TABLE 27
size, economic parameters and cost of each equipment for the process 2

| | | | | | Cost (k€) |
|---|---|---|---|---|---|
| | | Ref. | Ref. price | | of the |
| Equipment | Size | size | (k€) | Elasticity | equipment |
| QuenchSTR | 6 m³ | 3 m³ | 1,000 | 0.6 | 1,516 |
| Distill | 6 m³ | 3 m³ | 1,500 | | 2,274 |

| | |
|---|---|
| Total CAPEX | 3,789 |

It should be highlighted that, in the continuity of the use of generic separators, it is assumed that all these separators have the same investment cost parameters. This is obviously a strong W assumption, but it allows us at this stage to save a lot of time and information.

TABLE 28
total production cost (in € per kg of DVL in MAIN_0) with
breakdown of the fixed costs

| Item | Process 2 | Process 3 |
|---|---|---|
| CAPEX depreciation | 4.13 | 1.83 |
| Maintenance | 1.65 | 0.73 |
| Labour | 2.89 | 2.38 |
| Quality control | 0.20 | 0.39 |
| Overhead costs | 2.22 | 1.33 |
| Fixed cost subtotal | 11.09 | 6.65 |
| Variable cost subtotal | 31.94 | 28.61 |
| General total | 43.03 | 35.26 |

We could notice that the fixed costs amplify the advantage of the process 3 (based on thermal neutralisation of peroxide) in comparison with the process 2 (based on saline neutralisation). Hence, it is wise to focus the next investigations only on the process 3.

As regards the choice to use generic separators, it could be noted that, even though the contributions related to the equipment have been reduced to 0 €/kg in the case of the process 2, the process 3 would remain more advantageous. Hence, the choke of generic separators and the associated assumption did not distort the comparative conclusion.

If this choice had not been made, it would have been necessary to provide a large amount of information on the separators of the process 2 only to realise afterwards that this process had to be abandoned.

At this stage of the study, we have resorted to no kinetic parameters and to extremely few thermodynamic data. Moreover, only the separation by thermal neutralisation of peroxide and distillation of the acetic acid is still considered.

In other words, from the perspective of species-related parameters, it is possible to set out the following:

a bottom-up approach would have required from the outset collecting abundant data on each of the 12 species of Table 1, As well as on how they could interact.

the top-down approach disclosed herein enables us to reduce the list of species to 6 entries (including no saline species) before any collection of physico-chemical data, except for molar masses and normal boiling temperatures.

In the same way, the bottom-up approach would have required a study of the reactions of neutralisation of peroxide by sulphite and of neutralisation of acetic acid by carbonate. The top-down approach makes this study useless.

The models BOP1 and MEQ1 used so far have enabled us to sort out the configurations with a modest investment in terms of time and money. Nonetheless, for the configuration that appears to be the best, these models do not replace a study with conventional chemical engineering models (MEQ2 library).

At this stage, we have pulled out most advantage of the GUESS method. It is now time to focus on the weak points or inaccuracies that might impact the performances of the process.

During step 3.4, the operating parameters are determined by the system according to a standard model (MEQ2).

The next stage of the study would lead to three axes of improvement:

a description of the kinetics of the thermal neutralisation of the peroxide, which will allow to decide on the duration necessary for this reaction

the choice of a distillation piece of equipment, which will allow assessing the investment costs ore accurately

a description of the distillation by the laws of thermodynamic equilibrium, which will allow for a finer assessment of the quality of the separation.

During step 3.5, the operating parameters are determined by the system according to a detailed model (MEQ3).

It would be possible to further refine the study by resorting to models from the MEQ3 library. These models require the use of equipment from the BEQSPEC library because they use the geometric description of the equipment in order to decide, for example, on non-ideality of mixing or the formation of hot spots.

### Example 2

A second example of use of a system according to embodiments is given hereinafter, with reference to the method described in the U.S. Pat. No. 6,444,854.

The general objective is to minimise the production cost of enantiomerically pure or optically enriched sertraline-tetralone (in the R form) from a mixture containing two enantiomers (R and S) by means of chromatography such as chromatography on simulated moving bed (SMB).

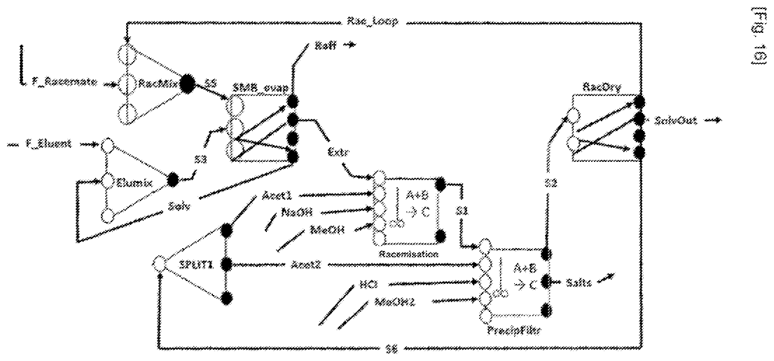

This general objective is declined into a series of questions: is it profitable to recycle the undesirable enantiomers in a racemisation reactor for mixing the racemate thus obtained with the fresh injection product (cf. FIG. 15)? Which elements of the process require an advanced study to ensure a good estimate of the costs and then of the relevant technical choices? In other words, where are the “bottleneck” information?

The only information available on the studied process originates from the aforementioned patent. This document does not provide any fundamental information on: the general thermodynamics, the sorption equilibriums, the kinetics of the racemisation reaction.

The available information consists of a short series of experience reports indicating what has been injected into which equipment and providing some macroscopic information on the observed consequences. The information contained in this document may also contain certain errors and should be treated with caution.

A conventional optimisation bottom-up approach would require a long and costly campaign of experiments to collect the missing fundamental information (thermodynamics, sorption, kinetics of the racemisation reaction in particular) for the description of SMB chromatography and of the racemisation reactor through conventional process engineering models.

This approach would be far too complex, long and costly to set up in the context of first assessment.

We apply hereinafter a top-down approach according to the invention. For illustration, this approach is used to compare two exemplar flow diagrams:

a diagram without recycling of the S-enantiomer, and without racemisation,

a simple diagram with recycling of the S-enantiomer, and with racemisation

In the first step, for Step 1: definition of the raw materials and of the species

For the diagram without recycling, the only raw materials are the racemic mixture and the fresh solvent. In case of racemisation, acetonitrile, methanol, soda and hydrochloric acid should be added.

Finally, the used raw materials and the associated species are listed in Table 29. To these 8 injected species, NaCl is added which might be generated in the process

TABLE 29
raw materials and associated species

| Raw materials | Associated species |
|---|---|
| Sertraline-tetralone racemate | R and S |
| Fresh solvent | 10% MeOH and 90% CH3CN |
| Acetonitrile | CH3CN |
| Methanol | MeOH |
| NaOH | NA+; OH— |
| HCl | H+; Cl— |
| Namely 6 raw materials | Namely 8 species |

In the second step of defining the operation block diagrams and in the context of the proposed top-down approach, the representation of the processes starts with the establishment of operation diagrams (step 2.1).

The two considered operation diagrams are represented.

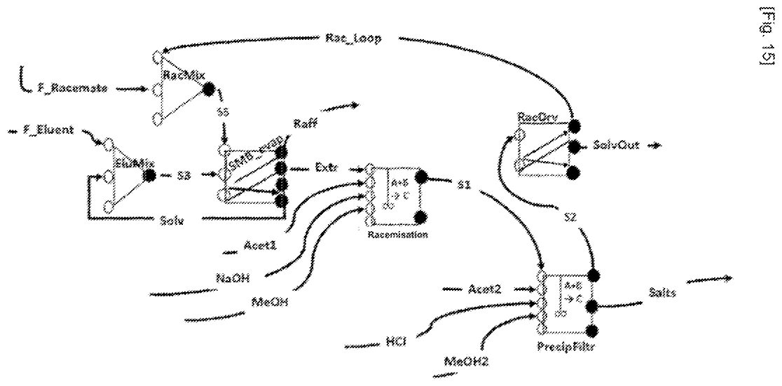

In FIG. 14: simple diagram, without recycling of the S-enantiomer, and without racemisation.

In FIG. 15: diagram with recycling of the S-enantiomer, and with racemisation.

The operation block diagrams could be read in flow (kg/h) as well as in amount (kg) treated per operation.

In step 2.2, the relationships between the inputs and the outputs of the operation blocks are defined.

The data from the U.S. Pat. No. 6,444,854 B1 and some reasonable assumptions enable us to describe the behaviour of each operation of FIGS. 14 and 15. Each operation is described by its effect on the flows (or amounts).

The operation block diagram of FIG. 14 contains only two operation blocks: a mixing operation block EluMix and a separation-evaporation operation block SMB_Evap which allows separating the enantiomers and concentrating them.

The operation block diagram of FIG. 15 contains the operations of FIG. 14 and also the following blocks.

Another mixing operation block RacMix.

A racemisation reaction operation block Racemisation: it converts the S-enantiomer into the R-enantiomer. The transformation stops when a racemic mixture is obtained.

An operation block PrecipFiltr.

A racemate drying operation block RacDry.

We will now detail the functioning of these different operations in terms of the relationships between the inputs and the outputs of the operation blocks.

The mixers EluMix and RacMix receive the input materials at 25° C.

The output compositions and temperature are a simple linear combination of the input ones; the mixtures are herein supposedly ideal liquids.

For the separation operations, separation ratios describe how a compound contained in the input flows is distributed between the output flows. The temperature, the state, the split ratios are given (possibly guessed/intuited/desired) by the user. They a either 0% or 100% in the tables hereinbelow but could take on any value comprised between 0% and 100% (cf. Table 33). The values are based on the chemist's experience or intuition or by some results from the U.S. Pat. No. 6,444,854 B1.

The operation block PrecipFiltr converts Na+ and Cl− (dissolved) into solid NaCl which is sent to SaltsOut. (cf. Table 30):

TABLE 30
separation ratios of the
operation PrecipFiltr

| | SaltsOut | S2 |
|---|---|---|
| NaCl (Solid) | 100% | 0% |
| Other species | 0% | 100% |

The operation block RacDry is described with the parameters of Table 31. They describe a perfect racemate drying operation. For this separation, the entire load is brought to 85° C. with evaporation of SolvOut (state change enthalpy of 850 kJ/kg) then the two outputs are brought back to 25° C. in the liquid state.

TABLE 31
separation ratios of the
operation block RacDry

| | SolvOut | Rac_Loop |
|---|---|---|
| R-Sertraline-tetralone | 0% | 100% |
| S-Sertraline-tetralone | 0% | 100% |
| Other species | 100% | 0% |

The performance of the SMB separation could be computed from the experimental results disclosed In the patent. We use data of the third experiment (cf. Table 32).

TABLE 32
SMB performances reported during the
third experiment (U.S. Pat. No. 6,444,854).

| | |
|---|---|
| Purity of the least retained enantiomer (%) | 99.7 |
| Recovery yield of the least retained enantiomer (%) | 98.4 |
| Computed necessary eluent volume | 0.40 |
| (L/g of enantiomers) | |

From this information, we could compute the separation ratios of the operation SMB_evap (cf. Table 33). We consider that the operation SMB_evap includes the chromatographic process, but also evaporation to remove a large part of the solvents. These evaporated solvents are recycled for economic reasons. For this purpose, the entire load of the operation is brought to 85° C., which vaporises the flow Solv (state change enthalpy of 850 kJ/kg). Afterwards, the three output flows are brought back to 25° C. in the liquid state.

TABLE 33
Separation ratios of the operation
block SMB_evap in the basic case

| | Raffinate | Extract | Solvent |
|---|---|---|---|
| R-Sertraline-tetralone | 98.4% | 1.6% | 0% |
| S-Sertraline-tetralone | 0.12% | 99.88% | 0% |
| Methanol | 0.5% | 0.5% | 99% |
| Acetonitrile | 0.5% | 0.5% | 99% |

During step 2.3, the overall balances are determined. The input flows are sized to obtain a flow rate of 12.5 kg/h (i.e., 100 tons a year for 8,000 hours) of R-enantiomer in the flow Raffinate.

The flow rate of the flow FreshEluent is computed so that the flow S3 meets the conditions of Table 3.

In the case without racemisation, we obtain the input flow rates disclosed in Table 34. The thermal power to be brought to the process amounts to 1042 kW, the power to be drawn has the same value.

TABLE 34
description of the output, input and internal flows
of the process in the case without recycling of the
S-enantiomer, and without racemisation.

| | Flow | Raw materials | Flow rate/Amounts | |
|---|---|---|---|---|
| OUTPUTS | Raffinate | | 32.58 | kg/h (including |
| | (containing | | 12.5 | kg/h of R) |
| | R-enantiomer) | | | |
| | Extract | | 32.96 | kg/h |
| INPUTS | FreshRacemate | Racemic mixture of enantiomers | 25.41 | kg/h |
| | FreshEluent | 90/10 acetonitrile methanol mixture | 50.8 | L/h |
| INTERNALS | S3 | | 4,013 | kg/h |
| | Solvent | | 3,973 | kg/h |

In case of recycling and racemisation of the S-enantiomer (cf. FIG. 15), the flow (amount) FreshRacemate is significantly reduced (cf. Table 35).

The amounts of raw materials necessary for the operation Racemisation (C2H1N_feed1, NaOH feed, MeOH feed, MeOH_feed2, C2H3N_feed2, HCl_feed) and the flow rates are computed based on the racemisation experiment described in the U.S. Pat. No. 6,444,854 to keep the proportions between these raw materials and the amount of enantiomers to be racemised. In particular, the flow rate (amount) of acetonitrile to be supplied is equal to 30 litres for each kilogram of S-enantiomer that is fed to the racemisation. The flow (amount) Extr that reaches the operation Racemisation contains acetonitrile that is deducted from the feed C2H3N_feed1.

The performance parameters of the SMB chromatography (cf, Table 33) are not affected by the racemisation.

In this second case, the thermal power to be brought to the process amounts to 1182 kW;

the thermal power to be drawn amounts 1131 kW.

TABLE 35
description of the inputs, outputs and internals
of the process in the case with recycling
of the S-enantiomer and racemisation.

| | | | Flow rate/ | |
|---|---|---|---|---|
| | Flow | Raw materials | Amounts | |
| OUTPUTS | Raffinate | | 32.65 | kg/h |
| | (containing | | | (including |
| | R-enantiomer) | | 12.5 | kg/h of R) |
| | Solv_Out | | 323.1 | kg/h |
| | Salt_Out | | 0.15 | kg/h |
| INPUTS | FreshRacemate | Racemic mixture of enantiomers | 12.52 | kg/h |
| | FreshEluent | 90/10 acetonitrile methanol mixture | 51 | L/h |
| | C2H3N_feed | acetonitrile | 106.88 | L/h |
| | NaOH_feed | NaOH | 0.1 | kg/h |
| | MeOH_feed | methanol | 0.07 | L/h |
| | MeOH_feed2 | methanol | 12.89 | kg/h |
| | C2H3N_feed2 | Acetonitrile | 257.76 | L/h |
| | HCl_feed | HCl | 1.94 | kg/h |
| INTERNALS | S3 | | 4,029 | kg/h |
| | S5 | | 25.42 | kg/h |
| | Extr | | 33.04 | kg/h |
| | Solvent | | 3,989 | kg/h |
| | S1 | | 117.6 | kg/h |
| | S2 | | 336.0 | kg/h |
| | RacLoop | | 12.90 | kg/h |

It should be noted that the results of Table 34 and Table 35 are obtained by taking into account the relationships between the inputs and outputs of each operation block and the connections between the operation blocks and that this consists of a first simulation computation result.

The system could now use the result of the simulation (i.e., the flows or amounts circulating in the processes given in Tables 34 and 35) to determine a first assessment of the variable production costs. For the economic assessment, we consider the following assumptions that are derived from experience. These are orders of magnitude:

the prices of the raw materials are those listed in Table 36

the solvent outputs are considered as wastes with a treatment cost of 0.15 €. The cost of the treatment of the other secondary outputs (Extr for the process without racemisation, SaltsOut for the process with racemisation) is neglected.

for the heat exchanges, a cost of 0.10 €/kWh is considered when it comes to energy to be brought to the process (heating/evaporation) and of 0.05 €/kWh when this energy has to be drawn (cooling/condensation).

TABLE 36
prices of the raw materials

| | Cost |
|---|---|
| Raw materials | (€/kg) |
| Sertraline-tetralone racemate | 100 |
| Acetonitrile | 20 |
| Methanol | 2 |
| 90/10 acetonitrile- | 19 |
| methanol mixture | |
| NaOH | 0.43 |
| HCl | 0.75 |

The information contained in Tables 34 to 36 leads to the variable costs of the two versions of the process disclosed in Table 37.

TABLE 37
contribution of each input to the variable part of the
R-enantiomer production cost, The values are
expressed in €/kg of R-enantiomer in the flow Raff.

| | | | Process 3 |
|---|---|---|---|
| | Process 1 Without racemisation | Process 2 With recycling of the S- enantiomer and racemisation | With recycling of the S″ enantiomer, racemisation and recycling of the acetonitrile |
| FreshRacemate | 203.25 | 100.12 | 100.12 |
| Total Acetonitrile | — | 461.10 | — |
| (two feeds) | | | |
| Total Methanol | — | 2.07 | 2.07 |
| (two feeds) | | | |
| Fresh eluent | 60.99 | 61.27 | 61.27 |
| NaOH_feed | — | <0.01 | <0.01 |
| HCl_feed | — | 0.12 | 0.12 |
| Treatment of the | 0.00 | 3.88 | 0.46 |
| effluents | | | |
| Energy | 12.51 | 13.58 | 15.68 |
| Total (€/kg of R | 276.75 | 642.14 | 190.53 |
| in Raffinate) | | | |

We can notice that the recycling and the racemisation (process are strongly penalised by the consumption of acetonitrile which is used as a reaction solvent.

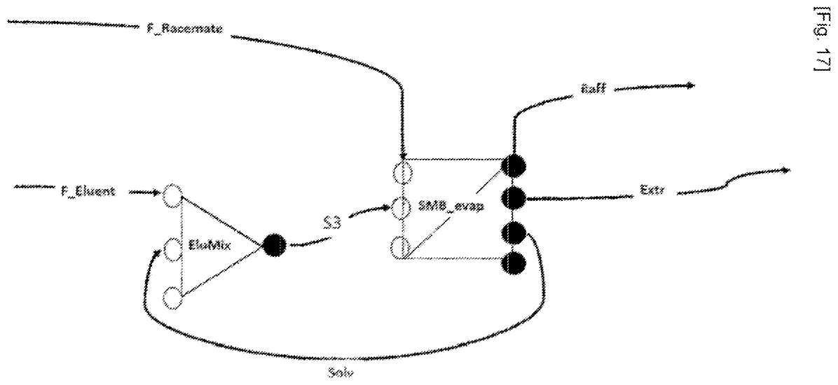

A purification and a partial recycling of this acetonitrile (cf. FIG. 16 could be considered. We use the previous methodology to assess this third option. It is shown that with 94% recycling of acetonitrile, no addition of acetonitrile is necessary, but the energy cost is increased by 2.10 € per kg of R-enantiomer in Huff. The total variable cost is equal to 190.53 €/kg, which is more attractive than the other options.

With this first approach, we have shown that option 2 is very likely to be unattractive from an economic perspective and in terms of waste production. Next, we will only consider options 1 and 3.

Afterwards, the definition of the equipment diagrams is described with reference to the various substeps already mentioned hereinbefore.

In step 3.1, each of the operation blocks of the previous diagrams is assigned to a piece of equipment.

Table 38 provides a correspondence between the operation blocks and the equipment. The generated equipment diagrams are illustrated in FIGS. 9 and 10. All of the equipment used herein belongs to the BEQGEN library.

TABLE 38
correspondence between operation blocks and equipment. The types
of equipment correspond to those defined in the BEQGEN library.

| Operation block | equipment (name) | equipment (type) | Volume (m³) |
|---|---|---|---|
| RacMix | RacemateMixer | Mixer | 2.5 |
| EluMix | EluentMixer | Mixer | 4.6 |
| Racemisation | RacemisationReactor | Reactor | 6 |
| PrecipFiltr Split1 | | | |
| RacDry | RacemateDryer | Separator | 6 |
| SMB_evap | SMB_evap | Separator | 0.3 |

For this example, an operation is assigned to a piece of equipment (Table 3S), except for RacemisationReactor which groups together several operations.

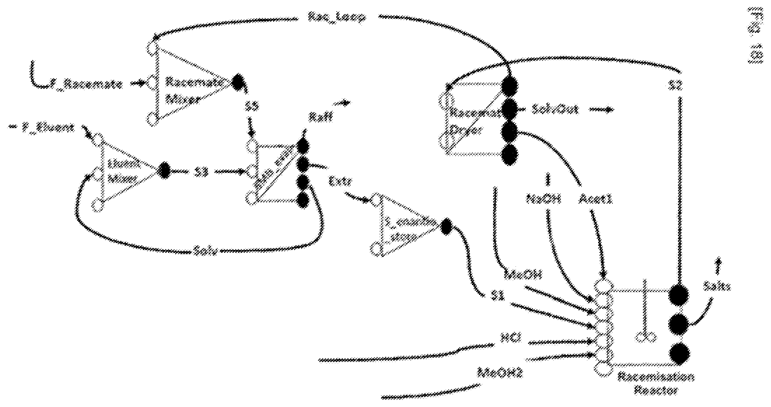

In the case of the diagram of FIG. 17, the two pieces of equipment operate continuously with the power supplies disclosed in Table 34.

In the case of the diagram of FIG. 18, part of the process is operated continuously (EluentMixer and SMB_evap) whereas another part of the process operates discontinuously (RacemisotionReactor and RacemateDryer) on cycles of 20 hours (including 10 hours of reaction). These cycles follow a procedure of the same nature as that disclosed in Table 22 and Table 23 of the previous example. The procedure of this second case study will not be detailed herein.

Hence, the equipment RacernateMixer and S_enantio_store must be able to store the production of one cycle. This second storage equipment is created for practical necessity, it has no equivalent in the operation diagram because the approach that is used has so far allowed the omission of the continuous-discontinuous interfacing constraints.

The operations, performed in the equipment of the two studied processes are described according to models from the MEQ1 library. These models follow the same guidelines as those of the MOP1 library (no use of physico-chemical data, macroscopic description of the phenomena) as well as the same structure. Therefore, the partition ratios of Tables 30 to 33, as well as the description of the racemisation reaction, keep all validity thereof. The input and output material and energy balances are therefore unchanged (cf. Table 34 for the case without racemisation).

Unlike the operation diagrams, the equipment diagrams manage the notions of time and equipment size. Thus, it is possible to access operating parameters such as the maximum filling rate or the productivity of either equipment. The values of these operating parameters enable the user to judge the practical relevance of the equipment sizes and the operating times he has specified. In the present case, given the initial sizes given in Table 38, the maximum occupancy rate of the racemisation reactor and of the racemate dryer is about 1303%©, which is obviously unrealistic. Similarly, for the SMB_evap, we would end up with a productivity of 42 kg of R-enantiomer per hour and per m³, whereas the current values for such separations are rather in the range of 25 kg of R-enantiomer per hour and per m′. As regards the racemisation, the document U.S. Pat. No. 6,444,854 reports an experiment where 100 g of S-enantiomer are treated in 6 hours in a volume of 3 to 5 litres of solution. This reaction, as conducted in the context of this experiment, therefore has a productivity of 3-4 kg_{s,input}/(h.m³_{container}).

An iterative process allows adjusting the sizes of the equipment so that the resulting operating parameters reach values that meet expectations. Thus, the sizes and operating parameters disclosed in Table 39 are reached.

In particular, it is noticed that, for the racemisation reactor, the constraint on the filling rate imposes operating at a productivity lower than that authorised by the chemical phenomena.

TABLE 39
dimension of the main equipment for a production of 12.5 kg/h of R-enantiomer; the possible
racemisation being performed in cycles of 20 hours

| Equipment | Vol. (m³) | Parameter | Value | |
|---|---|---|---|---|
| SMB_evap | 0.5 | Productivity | 25 | kg_{R}/(h.m³) |
| RacemateDryer | 10 | Filling | about 80% | |
| RacemisationReactor | 10 | Filling | about 80% | |
| | | Productivity | 2.5 | kgs,ᵢₙₚᵤₜ/ |
| | | | | (h.m³_{container}) |

| | |
|---|---|
| RacemateMixer | Not considered |
| EluentMixer | |
| S_enantio_store | |

At this stage, we have the sizing of all of the equipment essential for the production.

The system could use this information to perform a first economic estimate of the fixed costs.

The costs of the raw material are those in Table 36. The assumptions on the variable costs remain the same as before.

The method for computing the cost of the equipment is exactly the same as in Example 1. With the information given in Table 40, it is therefore possible to estimate the cost of the different equipment.

TABLE 40
size, economic parameters and cost of each equipment;

| | | | | | | Cost |
|---|---|---|---|---|---|---|
| | | | | | | (k€) |
| | | Size | | Ref. price | | of the |
| Equipment | Size | unit | Ref. size | (k€) | Elasticity | equipment |
| SMB__evap | 0.5 | m³ | 1 | 30,000 | 0.6 | 19,793 |
| RacemisationReactor | 10 | m³ | 3 | 1,000 | | 2,060 |
| RacemateDryer | 10 | m³ | 3 | 1,500 | | 3,090 |

| | |
|---|---|
| Total CAPEX (with neither racemisation nor acetonitrile recycling) | 19,793 |
| Total CAPE (with racemisation and acetonitrile recycling) | 24,940 |

As in Example 1, the contribution of the equipment to the production cost is computed while considering that the investment cost of the equipment is amortised over 64,000 hours of use. We also consider a maintenance cost that is equivalent to 5% of CAPEX per year (i.e., 8,000 hours).

In the present case, the labour cost is considered to be secondary (for the purpose of illustration) but could be considered. We consider a quality control cost of 100,000 €/year and an overhead cost that is equivalent to 25% of the other fixed costs. As regards the cost of the adsorbent used for chromatography in SMB, we consider a purchase cost of 10,000 €/kg, a duration of use of 16,000 hours and a mass of 1 kg per kg of R-enantiomer in Raff per day. For the case involving racemisation, it is considered that the process is stopped 10 hours between the cycles of 20 hours.

TABLE 41
total production cost (in € per kg of R-enantiomer
in Raff) with breakdown of the fixed costs

| | | Option 3 |
|---|---|---|
| | Option 1 Without racemisation | With racemisation of the S-enantiomer and acetonitrile recycling |
| Depreciation of the equipment | 24.74 | 46.79 |
| Maintenance | 9.89 | 18.72 |
| Absorbent | 15.00 | 15.00 |
| Quality control | 1.00 | 1.50 |
| Overhead costs | 12.66 | 20.50 |
| Total of the fixed costs | 63.29 | 102.51 |
| Total variable costs | 276.75 | 190.53 |
| Total (€/kg of R in Raffinate) | 340.04 | 293.04 |

Because of the presence of additional equipment, option 3 has higher fixed costs than option 1, Nonetheless, by combining variable and fixed costs, option 3 seems to be more attractive. In the following studies, only this configuration will be considered.

The preliminary computations presented before have raised the idea of a process configuration including the racemisation of the S-enantiomer and the recycling of the racemisation solvent. These computations have also indicated that, for the considered production scale, this configuration was the most interesting, which allows excluding the other two.

The approach proposed in the invention allows testing the impact of some macroscopic performance parameters to determine those that have the highest impact on the production cost. In the present case, this study is quite simple, Indeed, Table 39 indicates that, even with chemical phenomena that are a little slower or much faster than estimated, the size of the racemisation reactor would not be modified since it is fixed by the constraint of the filling rate. Hence, this would have no impact on the costs.

Conversely, the performance of the SMB (“Simulated Moving Bed”) has a direct impact on the costs through the eluent consumption, the amount of adsorbent or even the size of the SMB which is directly dependent on the productivity.

We could immediately see that the performances of SMB_evap has a much greater impact than that of the racemisation. It follows that, to have a better understanding of the process, we must focus our studies on the 5 MB rather than racemisation.

By using very simple diagrams and models, we have been able to simulate different process options and converge towards a reasonable option without any physico-chemical knowledge. We have identified that the SMB_evap is a particularly important piece of equipment at the economic level. If the project is of interest, it is obvious that we cannot content with the Guess level but that mechanistic/predictive models must be used to confirm, specify, or even invalidate the Guess level hypothesis. Conversely, performing another study of the racemisation at this stage would be a waste of time and money.

For a more advanced study of SMB_Evap, the use of mechanistic models (MEQ2 library) that require the determination of physico-chemical parameters is necessary. Where appropriate, it might even prove necessary to use models from the MEQ3 library that are capable of finely simulating the behaviour of a given piece of equipment. As regards the operation of SMB, a MC-LDF type model would certainly be recommended (Chromatographic Processes: modelling, simulation and design, Roger-Marc Nicoud, Cambridge University Press, 2015).

The approach we propose allowed us to choose a good configuration without requiring measurement of physico-chemical parameters. Now, to go further, we need these physico-chemical parameters, yet we know precisely which ones and why we have to measure them.

The invention's top-down approach starts with moderately complex and information-efficient computations, Thanks to the results of these first computations, we know which refinements have priority, which additional information should be

The technical effect of the system according to the invention is therefore to enable the obtainment of an equipment diagram of an industrial installation, then the completion and exploitation thereof. Thanks to the invention, this equipment diagram could be obtained more quickly than with traditional methods. Furthermore, thanks to the iteration options, with the improvement of the most critical elements, the obtained equipment diagram could feature better operating parameters than those obtained with traditional methods. The method and the device of the invention can be used by a laboratory chemist, who does not necessarily have the skills and experience of a process engineer.

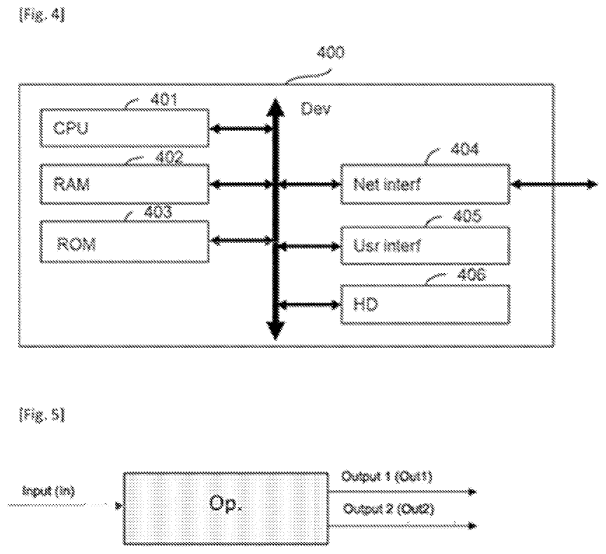

The various parts of the system described hereinabove may be implemented by one or several computer program(s). Thus, each module may correspond to a routine of one or several computer program(s). The system is then implemented by a global device 400 as illustrated in FIG. 4.

The device 400 comprises a communication bus connected to:

a central processing unit 401 such as a microprocessor, also denoted CPU;

a random-access memory 402, also denoted RAM, for storing an executable code of the method of the embodiments of the invention as well as the registers suited for recording the variables and the parameters necessary for the implementation of the method in accordance with the embodiments, the capacity of the memory could be increased by an optional RAM connected for example to an expansion port;

a read-only memory 403, also denoted ROM, for storing the computer programs used to implement the embodiments of the invention;

a network interface 404, which is typically connected to a communication network over which digital data to be processed is transmitted or received. The network interface 404 could be a single network interface or be composed of a set of different network interfaces (for example, wired and wireless interfaces, or different kinds of wired or wireless interfaces). The data is written on the network interface for transmission or read from the network interface for reception under the control of the software application running in the CPU 401;

a user interface 405 for the reception of the inputs of a user or for the display of information to the user;

a hard disk 406 also denoted HD

an input/output module 407 (also denoted I/O) to send/receive data from/towards devices such as a video source or a display screen.

The executable code could be stored either in the read-only memory 403, or on the hard disk 406, or on a removable digital medium such as a disk for example. According to one variant, the executable code of the programs could be received by means of a communication network, via the network interface 404, in order to be stored on one of the storage media of the communication device 400, such as the hard disk 406, before being executed.

The central processing unit 401 is adapted to control and direct the execution of the instructions or software code portions of the program in accordance with the embodiments of the invention, which instructions are stored on one of the aforementioned storage media. After being commissioned, the CPU 401 is able to execute the instructions from the main RAM memory 402 in connection with a software application for example after these instructions have been charged from the ROM program 403 or on the hard disk (HD) 406. This software application, when executed by the CPU 401, causes the method steps to be implemented in accordance with the embodiments.

### Example 3

Next, an exemplar simulation of a reaction operation (Op.) with separation of the downstream products is described as illustrated in FIG. 5. This simulation is carried out according to the principles described in detail hereinabove. The reaction is as follows: S1+S3→S2+S4 with total consumption of S3. The reactor input amounts are given by Table 1.

TABLE 1

| Species | Input flow (In) in moles |
|---|---|
| S1 | 10 |
| S2 | 0 |
| S3 | 5 |
| S4 | 0 |

Downstream of the reaction, the species are distributed between two outputs, denoted Out1 and Out2, according to the split ratios given in Table 2.

TABLE 2

| Species | Split ratio in the first output flow (Out1) | Split ratio in the second output flow (Out2) |
|---|---|---|
| S1 | 10% | 90% |
| S2 | 5% | 95% |
| S3 | 50% | 50% |
| S4 | 95% | 5% |

The input, first output and second output state vectors contain the numbers of moles of the different species and are respectively:

$${Y}_{E} = ( \begin{matrix}{m}_{S 1}^{In} \\ {m}_{S 2}^{In} \\ {m}_{S 3}^{In} \\ {m}_{S 4}^{In}\end{matrix} ) , {Y}_{S 1} = ( \begin{matrix}{m}_{S 1}^{Out 1} \\ {m}_{S 2}^{Out 1} \\ {m}_{S 3}^{Out 1} \\ {m}_{S 4}^{Out 1}\end{matrix} ) , {Y}_{S 2} = ( \begin{matrix}{m}_{S 1}^{Out 2} \\ {m}_{S 2}^{Out 2} \\ {m}_{S 3}^{Out 2} \\ {m}_{S 4}^{Out 2}\end{matrix} )$$

We obtain the foliowing explicit algebraic equations (Ex1, Ex2):

$${A}_{S 1} = ( \begin{matrix}0.1 & 0 & - 0.1 & 0 \\ 0 & 0.05 & 0.05 & 0 \\ 0 & 0 & 0 & 0 \\ 0 & 0 & 0.95 & 0.95\end{matrix} ) ; {Y}_{S 1} = {A}_{S 1} {Y}_{E} \qquad ( Ex 1 )$$

$${A}_{S 2} = ( \begin{matrix}0.9 & 0 & - 0.9 & 0 \\ 0 & 0.95 & 0.95 & 0 \\ 0 & 0 & 0 & 0 \\ 0 & 0 & 0.05 & 0.05\end{matrix} ) ; {Y}_{S 2} = {A}_{S 2} {Y}_{E} \qquad ( Ex 2 )$$

In this example, it is considered that during the separation the species are conveyed before their exit towards two perfectly homogeneous cells whose numbers of moles of the different species are grouped together in the vectors gₜ and X₂. It is also considered that the material withdrawn from the cells through the outputs (Out1 and Out2) has the same composition as that of the cells. It is then possible to write:

Yₛ₁=\tilde{X}₁ and Yₛ₂=\tilde{X}₂.

It is possible to transform the system (Ex1)(Ex2) into a differential system by introducing pseudo internal state variables (\tilde{X}₁ and \tilde{X}₂) and by setting:

$$- θ \frac{d {\tilde{X}}_{1}}{dt} - {\tilde{X}}_{1} + {A}_{S 1} {Y}_{E} = 0 and \qquad ( Ex 3 )$$

$$- θ \frac{d {\tilde{X}}_{2}}{dt} - {\tilde{X}}_{2} + {A}_{S 2} {Y}_{E} = 0 ; \qquad ( Ex 4 )$$

where θ is the arbitrary time constant of the homogeneous cells; when t becomes much greater than θ the differential term becomes zero and the pseudo state variables converge towards:

\tilde{X}₁=A_{S1} Y_{E} and \tilde{X}₂=A_{S2}Y_{E}

To find out (Ex1) and (Ex2), all itneed's is to set:

Y_{S1}=\tilde{X}₁ and Y_{S2}=\tilde{X}₂.

By choosing a low time constant, it is thus possible to switch from the explicit algebraic equations (Ex1 and Ex2) into differential equations (Ex3 and Ex4). These differential equations, involving pseudo internal state variables, could then be introduced into the system of differential equations simulating the entirety of the chemical or biochemical process.

1-7. (canceled)

8. A system for simulating a chemical or biochemical process, comprising at least one reaction or a separation transforming at least one raw material, said system including:
a plurality of functional modules configured to carry out respective levels of simulation of said chemical or biochemical process, wherein:
at least one functional module enables a simulation using a differential-algebraic modelling based on an equation of conservation of species, and
at least one functional module enables a simulation using an algebraic modelling relating inputs and outputs and/or initial states and final states at least of said at least one reaction,
at least one storage module for storing experimental data relating to chemical species in a data structure usable by at least one functional module of said plurality,
a performance evaluation module configured to:
carry out performance estimates of said process based on the use of experimental data derived from the storage module and/or simulation results obtained by one of the functional modules of said plurality,
compare at least two performance estimates with each other or with experimental results,
wherein said process is defined by a set of files shared by all of the modules of the system, each file including a description of said at least one raw material and a description of a decomposition of said at least one raw material into chemical species,
wherein said files are the inputs and the outputs of said modules of the system, the decomposition into chemical species being preserved throughout the processing.

9. The system according to claim 8, further including an experimental data processing and analysis module allowing processing signals, performing statistical analyses, identifying model parameters.

10. The system according to claim 8, including a database module gathering physico-chemical characteristics for the chemical species, on the one hand and of composition, origin and/or cost for raw materials.

11. The system according to claim 8, including:
a database module gathering physico-chemical characteristics for the chemical species, on the one hand, and of composition, origin and/or cost for the raw materials, on the other hand,
a central module configured to generate a composition vector representing a decomposition of said at least one raw material into a plurality of chemical species,
a file structure enabling the different modules to have access either to the raw materials and to their database, or to the species and to their database, or to both depending on the needs.

12. The system according to claim 8, for the computer simulation of a chemical or biochemical process comprising at least one first chemical or biochemical operation; said system comprising:
a solver module configured for solving of differential-algebraic equations,
a reception module configured to receive a first explicit algebraic equation representing said first chemical or biochemical operation and relating a first input state vector representing initial chemical amounts of said first operation to a first vector of output state variables representing final chemical amounts of said first operation; and
a processing module configured to:
in a first differential-algebraic equation relating:
said first vector of input state variables; and
a first vector of internal state variables of said first operation as an unknown of the equation;
inject into said first differential-algebraic equation the expression of the vector of output state variables of said first operation according to said first explicit algebraic equation as a vector of internal state pseudo-variables of said first operation, the steady-state solution of said differential-algebraic equation thus obtained thus converging towards said first vector of output state variables according to the first explicit algebraic equation,
set a time constant of said differential-algebraic equation thus obtained as being lower than a characteristic time of said first operation, and
implement the solver module on the differential-algebraic equation thus obtained to compute said first vector of output state variables.

13. The system according to claim 12, further comprising a selection module configured to select a simulation mode and wherein the processing module is configured to carry out said injection according to a selected mode.

14. The system according to claim 12; for computer simulation of a chemical or biochemical process further comprising a second chemical or biochemical operation; wherein:
said reception module is further configured to receive a second differential-algebraic equation representing said second chemical or biochemical operation relating:
a second vector of input state variables representing initial chemical amounts of said second operation; and
a second vector of internal state variables of said second operation as an unknown of the equation;
the steady-state solution of said second differential-algebraic equation converging towards a second vector of output state variables representing final chemical amounts of said second operation,
said processing module is further configured
to merge said first and second differential-algebraic equations and
to implement the solver module on the merger of said first and second differential-algebraic equations thus obtained to compute a vector of output state variables of the process.
